(19) United States
(12) Patent Application Publication
(10) Pub. No.: US 2023/0323430 A1
(43) Pub. Date: Oct. 12, 2023

(54) METHODS AND COMPOSITIONS FOR LIGATION AND SAMPLE ANALYSIS

(71) Applicant: 10x Genomics, Inc., Pleasanton, CA (US)

(72) Inventor: Shankar SHASTRY, Pleasanton, CA (US)

(73) Assignee: 10x Genomics, Inc., Pleasanton, CA (US)

(21) Appl. No.: 18/132,298

(22) Filed: Apr. 7, 2023

Related U.S. Application Data
(60) Provisional application No. 63/329,320, filed on Apr. 8, 2022.

(51) Int. Cl. C12Q 1/682; C12Q 1/6874; C12Q 1/6855

(52) U.S. Cl. CPC C12Q 1/682; C12Q 1/6874; C12Q 1/6855

(57) ABSTRACT

The present disclosure relates in some aspects to methods for analyzing a target nucleic acid in a biological sample. In some aspects, provided herein are methods and compositions for improving the specificity of ligation in situ in biological samples, as well as in single cell analysis and spatial applications of RNA templated ligation reactions.

### CROSS-REFERENCE TO RELATED APPLICATION

This application claims priority to U.S. Provisional Patent Application No. 63/329,320, filed Apr. 8, 2022, entitled “Methods and Compositions for Ligation and Sample Analysis,” which is herein incorporated by reference in its entirety for all purposes.

### FIELD

The present disclosure relates in some aspects to methods and compositions for processing nucleic acid molecules, including improving ligation specificities during analysis of a sample, such as detection of a nucleic acid sequence in situ in a biological sample, in a cell (e.g., single cells of a cell population), within a partition, or on a substrate comprising spatially barcoded oligonucleotides.

### BACKGROUND

Methods are available for analyzing nucleic acids present in a biological sample, such as a cell or a tissue. The detection and analysis of nucleic acid sequences, specifically detection and analysis of the RNA transcriptome, has applications in many different fields, including cancer diagnosis, personalized medicine, and infectious diseases. However, current approaches suffer from reduced specificity and/or poor sensitivity. Improved methods for analyzing nucleic acids present in and/or from a biological sample with increased specificity and sensitivity are needed. The present disclosure addresses this and other needs.

### BRIEF SUMMARY

In some aspects, provided herein are methods and compositions for analyzing a target nucleic acid in a biological sample, wherein ligation of a probe or probe set hybridized to the target RNA molecule is blocked, unless a stem-loop structure present within the probe or probe set is cleaved, e.g., using a nuclease. In some aspects, also provided herein are methods for detecting a region of interest in a target nucleic acid (for example, a SNP in an RNA molecule). In some aspects, the presence, amount, and/or identify of a target nucleic acid is analyzed in situ in a biological sample (e.g., at a location in a cell or tissue sample), using an array of oligonucleotides comprising capture agents and spatial barcodes, or in single cells (e.g., using a single-cell assay comprising partitioning a single cell or compotents thereof in a partition). Also provided are polynucleotides, sets of polynucleotides, compositions, and kits for use in accordance with the methods, for example for RNA-targeting circularizable probe-mediated SNP detection.

Provided herein include methods and compositions for nucleic acid ligation, including DNA-templated ligation and/or RNA-template dependent ligation of DNA probes (e.g., RNA-templated ligation), and use of the methods and compositions for in situ detection and ex situ sequencing applications. In some embodiments, provided herein is a method for analyzing a target nucleic acid, comprising a) contacting the target nucleic acid with a probe or probe set, wherein the probe or probe set comprises: i) a first hybridization region capable of hybridizing to a first target sequence in the target nucleic acid, ii) a second hybridization region capable of hybridizing to a second target sequence in the target nucleic acid, and iii) a duplex region, wherein upon hybridization of the probe or probe set to the target nucleic acid, the duplex region is positioned between the first and second hybridization regions; b) cleaving the probe or probe set, e.g., with a nuclease, to generate a first ligatable end and release the duplex region or a portion thereof; c) ligating the first ligatable end to a second ligatable end in the probe or probe set (e.g., when the probe or probe set is hybridized to the target nucleic acid) to generate a ligated probe; and d) detecting the ligated probe or a product thereof. In some embodiments, the ligated probe or product thereof can be generated and detected in a biological sample (e.g., a cell or a tissue sample) comprising the target nucleic acid. In some embodiments, the ligated probe or product thereof can be generated in a partition (e.g., an emulsion droplet or microwell comprising a single cell or components of the single cell). In some embodiments, the ligated probe or product thereof can be sequenced using nucleic acid sequencing. In some embodiments, the ligated probe or product thereof can be generated in a biological sample (e.g., a cell or a tissue sample) comprising the target nucleic acid, and the ligated probe or product thereof can be captured by an oligonucleotide immobilized on a substrate and spatially barcoded for subsequent analysis, e.g., using nucleic acid sequencing. In some embodiments, the ligated probe or product thereof can be generated on a substrate and spatially barcoded. In some embodiments, the probe can be a circular probe (e.g., a circular oligonucleotide), wherein a circularizable probe is generated from the circular probe by nuclease cleavage. In some embodiments, the probe or probe set can be a circularizable probe or probe set (e.g., a circularizable oligonucleotide or oligonucleotide set). In some embodiments, the probe or probe set can be circularizable using the target nucleic acid and/or a splint as template. In some embodiments, the probe or probe set can comprise a first and second probe that can be ligated to form a linear probe (e.g., a non-circularized probe). In some embodiments, the duplex region can be at the juxtaposition between the first and second hybridization regions upon hybridization of the probe or probe set to the target nucleic acid.

In any of the embodiments herein, the duplex region can be 3′ or 5′ to the first hybridization region. In any of the embodiments herein, the duplex region can be 5′ or 3′ to the second hybridization region. In any of the embodiments herein, the duplex region can be between or at a 3′ end, internal, or at a 5′ end of the probe or a probe in the probe set. In any of the embodiments herein, the duplex region can be at the 5′ end or 3′ end of the probe or a probe in the probe set. In any of the embodiments herein, the duplex region can be at the 5′ end and 3′ end of the probe or the probe in the probe set. In any of the embodiments herein, the duplex region can be formed by the 5′ and 3′ ends of the probe. In any of the embodiments herein, the duplex region can be formed by a 5′ end and a 3′ end of a same probe or different probes in the probe set. In any of the embodiments herein, the probe or probe set can comprise a stem-loop structure comprising the duplex region. In any of the embodiments herein, the duplex region can be in the stem of the stem-loop structure. In any of the embodiments herein, the duplex region can be at least 5 nucleotides in length. In any of the embodiments herein, the duplex region can be at least about 4, at least about 6, at least about 8, at least about 10, at least about 12, at least about 14, at least about 16, at least about 18, or at least about 20 base pairs in length.

In any of the embodiments herein, the duplex region can comprise a first strand and a second strand.

In any of the embodiments herein, the first strand and the second strand can be part of the same probe molecule comprising self-complementary sequences. In any of the embodiments herein, the first strand can be at the 5′ end and the second strand can be at the 3′ end of the probe. In any of the embodiments herein, the first strand can be at the 3′ end and the second strand can be at the 5′ end of the probe. In any of the embodiments herein, the first strand and the second strand can be continuous with the first or second hybridization region of the probe. In any of the embodiments herein, the first strand and the second strand can be continuous, and the continuous strand can be at the 5′ end or at the 3′ end of the probe molecule.

In any of the embodiments herein, the first strand and the second strand can be part of two distinct probes. In any of the embodiments herein, the first strand and the second strand are in a first probe and a second probe, respectively. In any of the embodiments herein, the first strand can be at the 5′ end of the first probe and the second strand can be at the 3′ end of the second probe. In any of the embodiments herein, the first strand can be at the 3′ end of the first probe and the second strand can be at the 5′ end of the second probe.

In any of the embodiments herein, the probe set can comprise a first probe and a second probe, and the first strand and the second strand can be continuous and can be in the first probe or the second probe. In any of the embodiments herein, the first strand and the second strand can be at the 5′ end or at the 3′ end of the first probe. In any of the embodiments herein, the first strand and the second strand can be at the 5′ end or at the 3′ end of the second probe.

In any of the embodiments herein, in the cleaving step in b), a site in the duplex region can be cleaved. In any of the embodiments herein, one or both of the strands in the duplex region can be cleaved. In any of the embodiments herein, the cleaving can generate a nick on one strand of the duplex region, a blunt end, and/or a sticky end.

In any of the embodiments herein, the probe or probe set can further comprise a single-stranded region linking the duplex region to the first or second hybridization region, and in the cleaving step b), a site in the single-stranded region can be cleaved.

In any of the embodiments herein, the first ligatable end can be a 3′ ligatable end or a 5′ ligatable end, and second ligatable end can be a 5′ ligatable end or a 3′ ligatable end, respectively. In some embodiments, the cleaving in step b) only generates the first ligatable end. In some embodiments, the cleaving in step b) can generate both the first ligatable end and the second ligatable end. In any of the embodiments herein, the first ligatable end and the second ligatable end can be in the same oligonucleotide molecule or in different oligonucleotide molecules.

In any of the embodiments herein, the probe or probe set can comprise a non-ligatable end and the cleaving in step b) can remove the non-ligatable end. In some embodiments, the non-ligatable end may be a 3′ end or a 5′ end. In some embodiments, the non-ligatable end may comprise a 3′ dideoxynucleotide (ddNTP), may lack a 3′ hydroxyl group, or may be 5′ dephosphorylated. In some embodiments, the non-ligatable end may be a 3′ end or a 5′ end of the duplex region. In some embodiments, the non-ligatable end may comprise a 3′ dideoxy protected terminal. In some embodiments, the non-ligatable end may be a 3′ end or a 5′ end of a region separated from the first or second hybridization region by the duplex region.

In any of the embodiments herein, the ligated probe may be a linear probe or a circular probe. In any of the embodiments herein, the ligated probe can be generated using the target nucleic acid as template. In some embodiments, the method can further comprise amplifying the ligated probe. In some embodiments, the ligated probe can be amplified in situ in the biological sample. In some embodiments, the ligated probe can be amplified using rolling circle amplification (RCA). In any of the embodiments herein, an amplification product of the ligated probe can be detected in situ in the biological sample. In any of the embodiments herein, the method may further comprise detecting the released duplex region or portion thereof and/or a product thereof. In any of the embodiments herein, the released duplex region or portion thereof and/or the product thereof can be detected outside a cell or a biological sample.

In any of the embodiments herein, the target nucleic acid can be at a location in a biological sample. In any of the embodiments herein, the ligated probe can be generated at the location in the biological sample. In any of the embodiments herein, the ligated probe can be amplified at the location in the biological sample. In any of the embodiments herein, the ligated probe and/or the product thereof can be detected at the location in the biological sample. In any of the embodiments herein, the product can be an RCA product at the location in the biological sample, and the RCA product can be detected using one or more detectable probes, e.g., using fluorescently labeled probes that bind to the RCA product and/or using detectable probes that bind to the RCA product and fluorescently labeled probes.

In any of the embodiments herein, the target nucleic acid can be at a location in a biological sample and the ligated probe can be generated at the location in the biological sample, and wherein the ligated probe and/or the product thereof can be covalently or noncovalently attached to an oligonucleotide immobilized on a substrate. In some embodiments, the oligonucleotide can comprise a spatial barcode sequence. In some embodiments, the oligonucleotide can comprise a capture sequence complementary to a sequence of the ligated probe and/or the product thereof. In some embodiments, a spatially barcoded oligonucleotide comprising (i) a sequence of the ligated probe or product thereof or complement thereof and (ii) a sequence of the spatial barcode sequence or complement thereof can be generated and detected, e.g., by nucleic acid sequencing.

In any of the embodiments herein, the target nucleic acid can be covalently or noncovalently attached to an oligonucleotide immobilized on a substrate. In any of the embodiments herein, the ligated probe and/or the product thereof can be generated. In any of the embodiments herein, the ligated probe and/or the product thereof can be amplified on the substrate. In any of the embodiments herein, the oligonucleotide can comprise a spatial barcode sequence. In any of the embodiments herein, the ligated probe and/or the product thereof can comprise the spatial barcode sequence or a complement thereof. In any of the embodiments herein, the ligated probe and/or the product thereof can be detected by nucleic acid sequencing. In any of the embodiments herein, a spatially barcoded oligonucleotide comprising (i) a sequence of the ligated probe or product thereof or complement thereof and (ii) a sequence of the spatial barcode sequence or complement thereof can be generated and detected, e.g., by nucleic acid sequencing.

In any of the embodiments herein, the method can further comprise releasing the spatially barcoded oligonucleotide from the substrate. In some embodiments, the method can comprise sequencing the spatially barcoded oligonucleotide. In some embodiments, the target nucleic acid or sequence thereof is detected at the spatial location in the biological sample.

In some embodiments, the target nucleic acid can be in a partition. The partition can contain a single cell or components of a lysed single cell. In some embodiments, the ligated probe can be generated and in the partition. In some embodiments, the ligated probe can be amplified in the partition. In any of the preceding embodiments, the partition can be a microwell or a droplet. In some embodiments, the partition can be an emulsion droplet. In any of the preceding embodiments, the partition can comprise a support that comprises a plurality of barcode oligonucleotides comprising a partition barcode sequence. In any of the preceding embodiments, the barcode oligonucleotides can comprise a capture sequence. In some embodiments, the barcode oligonucleotides comprise additional unique molecular identifier (UMI). In any of the embodiments herein, the barcode oligonucleotides can be releasably attached to the support. In any of the preceding embodiments, the support can be a bead. In some embodiments, the support is a gel bead. In any of the preceding embodiments, the method can further comprise using the barcode oligonucleotides to generate a barcoded ligated probe molecule. In some embodiments, the barcoded ligated molecule may comprise (i) a sequence of the ligated probe or product thereof or a complement of the ligated probe or product thereof, and (ii) a sequence of the partition barcode sequence or complement thereof. In any of the preceding embodiments, the ligated probe and/or the product thereof can comprise the partition barcode sequence or a complement thereof. In any of the preceding embodiments, the method can further comprise releasing the barcoded ligated probe molecule from the partition. In any of the preceding embodiments, the method can further comprise pooling the barcoded ligated probe molecule from the partition with contents of other partitions of a plurality of partitions. In any of the preceding embodiments, the method can further comprise amplifying the barcoded-ligated probe molecule. In any of the embodiments herein, the ligated probe and/or the product thereof can be detected by nucleic acid sequencing.

The present disclosure in some aspects provides a method comprising providing a partition comprising (i) a single biological particle, wherein said single biological particle comprises target nucleic acid and the probe or probe set hybridized to the target nucleic acid, and (ii) a bead comprising barcode oligonucleotides comprising a partition barcode sequence and capture sequence complementary to a sequence of the ligated probe and/or product thereof; using the barcode oligonucleotides to generate a barcoded ligated probe molecule comprising (i) a sequence of the ligated probe or product thereof or complement thereof and (ii) a sequence of the partition barcode sequence or complement thereof; releasing the barcoded ligated probe molecule from the partition and amplifying the barcoded ligated probe molecule; and determining a sequence of the barcoded ligated probe molecule or a portion thereof.

The present disclosure in some aspects provides a method for analyzing a target nucleic acid, comprising: (a) providing a partition comprising (i) a single biological particle, wherein said single biological particle comprises the target nucleic acid and a probe or probe set hybridized to the target nucleic acid, and (ii) a bead comprising a plurality of barcode oligonucleotides each comprising a partition barcode sequence and capture sequence complementary to a sequence in the probe or probe set or a product thereof; wherein the probe or probe set comprises: i) a first hybridization region capable of hybridizing to a first target sequence in the target nucleic acid, ii) a second hybridization region capable of hybridizing to a second target sequence in the target nucleic acid, and iii) a duplex region, wherein upon hybridization of the probe or probe set to the target nucleic acid, the duplex region is positioned between the first and second hybridization regions; wherein the probe or probe set is cleaved with a nuclease to generate a first ligatable end and release the duplex region or a portion thereof; b) ligating the first ligatable end to a second ligatable end in the probe or probe set hybridized to the target nucleic acid to generate a ligated probe; and (c) using a barcode oligonucleoctide of the plurality of barcode oligonucleotides to generate a barcoded ligated probe molecule comprising (i) a sequence of the ligated probe or product thereof or complement thereof and (ii) a sequence of the partition barcode sequence or complement thereof; (d) releasing the barcoded ligated probe molecule from the partition and amplifying the barcoded ligated probe molecule; and (e) determining a sequence of the barcoded ligated probe molecule or a portion thereof. In some of the embodiments herein, the biological particle can be a cell. In some embodiments herein, step e) determining the sequence of the barcoded ligated probe molecule or a portion thereof can comprise performing next generation sequencing.

In any of the embodiments herein, the target nucleic acid can comprise RNA and the probe or probe set comprises DNA and/or RNA. In any of the embodiments herein, the target nucleic acid can be an mRNA. In any of the embodiments herein, the first ligatable end and/or the second ligatable end in the probe or probe set can comprise one or more ribonucleotide residues. In any of the embodiments herein, the first ligatable end or the second ligatable end can comprise a 3′ ribonucleotide residue. In any of the embodiments herein, the first ligatable end or the second ligatable end may comprise no more than four consecutive ribonucleotide residues.

In any of the embodiments herein, the nuclease can be a restriction endonuclease. In any of the embodiments herein, the restriction endonuclease may have a recognition sequence that is 4, 5, 6, 7, 8, or more base pairs in length.

In any of the embodiments herein, cleavage by the restriction endonuclease, either in or outside the recognition sequence, may generate blunt ends. In any of the embodiments herein, the restriction endonuclease may be selected from the group consisting of AanI, Acc16I, AccBSI, AcvI, AfeI, AjiI, Aor51HI, BalI, BmgBI, Bsp68I, BsrBI, BssNAI, Bst11071, BstSNI, BtuMI, DinI, DraI, Ecl136II, Eco105I, Eco147I, Eco321, Eco47III, Eco53kI, Eco721, EcoICRI, EcoRV, EgeI, EheI, FspI, HpaI, KspAI, MbiI, MluNI, Mox20I, MscI, Msp20I, MssI, NaeI, NruI, NsbI, PceI, PdiI, PmaCI, PmeI, PmII, PsiI, PspCI, PvuII, RruI, ScaI, SfoI, SmaI, SnaBI, SrfI, SseBI, SspI, StuI, SwaI, ZraI, and ZrmI.

In any of the embodiments herein, cleavage by the restriction endonuclease, either in or outside the recognition sequence, may generate sticky ends. In any of the embodiments herein, the restriction endonuclease may be selected from the group consisting of AatII, AbsI, Acc65I, AccIII, AcII, AfIII, AgeI, AhII, Alw44I, Aor13HI, ApaI, ApaLI, AscI, AseI, AsiGI, AsiSI, Asp718I, AspA2I, AsuII, AvrII, BamHI, BauI, BbvCI, BcII, BcuI, BfrI, BgIII, BlnI, BmtI, Bpu14I, Bsa29I, BseAI, BseCI, BsePI, BseX3I, BseYI, BshTI, BshVI, BsiWI, Bsp119I, Bsp120I, Bsp13I, Bsp1407I, Bsp19I, BspDI, BspEI, BspHI, BspMAI, BspOI, BspT104I, BspTI, BsrGI, BssHII, BssSI-v2, Bst2BI, BstAFI, BstAUI, BstBI, BstZI, Bsu15I, BsuTUI CciI, CciNI, Cfr42I, Cfr9I, ClaI, CspAI, EagI, EclXI, EcoRI, EcoT22I, FauNDI, FbaI, FseI, GsaI, HindIII, HpaI, KasI, Kpn2I, KpnI, Ksp22I, KspI, MauBI, MfeI, MluI, Mly113I, Mph1103I, MreI, MroI, MroNI, MspCI, MunI, NarI, NcoI, NdeI, NgoMIV, NheI, NotI, Nsil, NspV, PaeI, PaeR7I, PagI, PalAI, PauI, PciI, Pfl23II, PinAI, Ple19I, PluTI, PscI, PshBI, Psp124BI, Psp1406I, PspFI, PspLI, PspOMI, PstI, PteI, PvuI, RgaI, RigI, SacI, SacII, SaII, SbfI, SdaI, SfaAI, Sfr274I, Sfr202I, SfuI, SgfI, SgrBI, SgrDI, SgsI, SlaI, SpeI, SphI, Sse83871, SspDI, SstI, TspMI, Vha464I, VneI, VspI, XbaI, XhoI, XmaI, and Zsp2I.

In any of the embodiments herein, the restriction endonuclease may cleave in the recognition sequence and/or at a site outside of the recognition sequence. In any of the embodiments herein, the restriction endonuclease may be selected from the group consisting of AcuI, AlwI, BaeI, BbsI, BbsI-HF, BbvI, BccI, BceAI, BcgI, BciVI, BcoDI, BfuAI, BmrL, BpmL, BpuEI, Bsal-HFv2, BsaXI, BseRI, Bsgl, BsmAI, BsmBI-v2, BsmFI, BsmI, BspCNI, BspMI, BspQI, BsrDI, Bsrl, BtgZI, BtsCI, Btsl-v2, BtsIMutl, CspCI, EasI, EciI, Esp3I, FauI, FokI, Hgal, HpyAV, MboII, MIyI, MnII, NmeAIII, PaqCI, PIeI, SapI, and SfaNI.

In any of the embodiments herein, the nuclease may comprise a uracil-DNA glycosylase (UDG) and/or an endonuclease. In some embodiments, the nuclease may be an Endonuclease VIII. In any of the embodiments herein, the nuclease may comprise a uracil-specific excision reagent enzyme. In any of the embodiments herein, the nuclease may comprise a nickase. In any of the embodiments herein, the recognition sequence of the nickase may be selected from the group consisting of Nt.CviPII, Nb.BsmI, Nb.BbvCI, Nb.BsrDI, Nb.BtsI, Nt.BsmAI, Nt.BbvCI, NtBspQI, Nt.AlwI, and Nt.BstNBI. In some embodiments herein, the nickase is Nt.BbvCI. In some embodiments herein, the nickase is Nt.CviPII. In any of the embodiments herein, the method can further comprise a wash step to remove the released duplex region or portion thereof. In any of the embodiments herein, the wash step can be performed using conditions that allow the probe or probe set to remain hybridized to the target nucleic acid. In any of the embodiments herein, the method can further comprise detecting the released duplex region or portion thereof and/or a product thereof. In any of the embodiments herein, the released duplex region or portion thereof and/or the product thereof can be detected on an array (e.g., using probe hybridization) and/or by nucleic acid sequencing (e.g., after removing the duplex region or portion thereof and/or the product thereof from the array).

In any of the embodiments herein, the target nucleic acid can comprise a region of interest and the probe or probe set can comprise an interrogatory region. In any of the embodiments herein, the interrogatory region may be in the duplex region. In any of the embodiments herein, the interrogatory region may be in a stem-loop structure comprising the duplex region, and the interrogatory region may be in the stem and/or the loop of the stem-loop structure. In some embodiments, the interrogatory region in the stem-loop structure is not capable of hybridizing to the region of interest until the cleaving in step b).

In some embodiments, the interrogatory region is not capable of hybridizing to the region of interest until the cleaving in step b). In any of the embodiments herein, the cleaving in step b) may allow hybridization between the interrogatory region and the region of interest. In any of the embodiments herein, the interrogatory region can be at or near the first ligatable end. In some embodiments, the cleaving in step b) exposes the interrogatory region or a portion thereof, rendering it available for hybridizing to the region of interest. For example, the interrogatory region or portion thereof can be part of one strand of the duplex region hybridized to the other strand of the duplex region, whereby the interrogatory region or portion thereof is masked from hybridization to the target nucleic acid. In another example, the interrogatory region or portion thereof can be part of a stem-loop structure which prevents the interrogatory region or portion from hybridization and/or ligation.

In some embodiments, the interrogatory region is capable of hybridizing to the region of interest but the probe or probe set is not capable of being ligated to form the ligated probe until the cleaving in step b). In some embodiments, after the cleaving step in b), the interrogatory region is at or near the first ligatable end. In some embodiments, before and/or after the cleaving step in b), the interrogatory region is at or near the second ligatable end. In any of the embodiments herein, the interrogatory region may comprise a portion of the first ligatable end and a portion of the second ligatable end. In some embodiments, the first ligatable end and the second ligatable end together form the interrogatory region or a portion thereof. In any of the embodiments herein, the interrogatory region can be at or near a 3′ or 5′ ligatable end. In any of the embodiments herein, the interrogatory region can be no more than about 5, 4, 3, 2, or 1 nucleotide from a 3′ hydroxyl or a 5′ phosphate group of the first or second ligatable end.

In some embodiments, the region of interest and the interrogatory region can comprise a single nucleotide. In some embodiments, the interrogatory region and the region of interest can be a single nucleotide. In some embodiments, the region of interest is selected from the group consisting of a single-nucleotide polymorphism (SNP), a single-nucleotide variant (SNV), a single-nucleotide substitution, a point mutation, a single-nucleotide deletion, and a single-nucleotide insertion.

The present disclosure in one aspect provides a method for analyzing a target RNA, comprising: a) contacting the target RNA with a first probe and a second probe simultaneously or in any order, wherein the first probe comprises: i) a first hybridization region capable of hybridizing to a first target RNA sequence in the target RNA, and ii) a stem-loop structure, the second probe comprises a second hybridization region capable of hybridizing to a second target RNA sequence in the target RNA, and upon hybridization of the first and second probes to the target RNA, the stem-loop structure is at positioned between the first and second hybridization regions; b) cleaving the first probe with a nuclease to generate a first ligatable end, thereby releasing the stem-loop structure or a portion thereof; c) ligating the first ligatable end to a second ligatable end in the second probe to generate a ligated probe using the target RNA as template; and d) detecting the ligated probe or a product thereof.

In some embodiments, the target RNA is in a cell or tissue sample and the ligated probe and/or the product thereof is generated in the cell or tissue sample. In some embodiments, the ligated probe and/or the product thereof is detected in the cell or tissue sample.

In some embodiments, the ligated probe and/or the product thereof is covalently or noncovalently attached to an oligonucleotide immobilized on a substrate. In some embodiments, the oligonucleotide comprises a spatial barcode sequence and a spatially barcoded oligonucleotide comprising (i) a sequence of the ligated probe or product thereof or complement thereof and (ii) a sequence of the spatial barcode sequence or complement thereof is generated and sequenced.

In some embodiments, the target RNA is in a partition, and the partition comprises a single cell or components of the single cell containing the target RNA. In some embodiments, the partition is a microwell or a droplet. In some embodiments, the partition can be an emulsion droplet. In some embodiments, the ligated probe or product thereof is generated in the partition, and the partition further comprises a support that comprises a nucleic acid molecule comprising a partition barcode sequence. In some embodiments, the support is a bead. In some embodiments, the support can be a gel bead. In some embodiments, a barcoded oligonucleotide comprising (i) a sequence of the ligated probe or product thereof or complement thereof and (ii) a sequence of the partition barcode sequence or complement thereof is generated and sequenced.

In some embodiments, the target RNA is covalently or noncovalently attached to an oligonucleotide immobilized on a substrate and the ligated probe and/or the product thereof is generated. In some embodiments, the ligated probe and/or the product thereof can be amplified on the substrate. In some embodiments, the oligonucleotide comprises a spatial barcode sequence. In some embodiments, a spatially barcoded oligonucleotide comprising (i) a sequence of the ligated probe or product thereof or complement thereof and (ii) a sequence of the spatial barcode sequence or complement thereof is generated and sequenced.

The present disclosure in another aspect provides a method for analyzing a biological sample, comprising: a) contacting the biological sample with a circularizable probe comprising: i) a first hybridization region capable of hybridizing to a first target sequence in a target nucleic acid in the biological sample, ii) a second hybridization region capable of hybridizing to a second target sequence in the target nucleic acid, and iii) a stem-loop structure at the 3′ or 5′ end of the circularizable probe, wherein upon hybridization of the circularizable probe to the target nucleic acid, the stem-loop structure is positioned between the first and second hybridization regions; b) cleaving the circularizable probe with a nuclease to generate a ligatable 3′ end or ligatable 5′ end and release the stem-loop structure or a portion thereof; c) ligating the ligatable 3′ end or ligatable 5′ end to the 5′ end or the 3′ end of the circularizable probe, respectively, to generate a circular probe using the target nucleic acid as template; d) amplifying the circular probe using rolling circle amplification (RCA) to generate an RCA product; and e) detecting the RCA product in the biological sample.

In any of the embodiments herein, the stem of the stem-loop structure may be cleaved by the nuclease to generate the ligatable 3′ end or ligatable 5′ end in the cleaving step b).

The present disclosure in another aspect provides a method for analyzing a biological sample, comprising: a) contacting the biological sample with a circularizable probe comprising: i) a first hybridization region capable of hybridizing to a first target sequence in a target nucleic acid in the biological sample, ii) a second hybridization region capable of hybridizing to a second target sequence in the target nucleic acid, iii) a stem-loop structure at the 3′ or 5′ end of the circularizable probe, wherein the target nucleic acid comprises a region of interest and the stem-loop structure comprises an interrogatory region, and wherein upon hybridization of the circularizable probe to the target nucleic acid, the stem-loop structure is positioned between the first and second hybridization regions; b) cleaving the circularizable probe with a nuclease to generate a ligatable 3′ end or ligatable 5′ end and releasing the stem-loop structure or a portion thereof, thereby allowing the interrogatory region to hybridize to the region of interest; c) if the interrogatory region is complementary to the region of interest, ligating the ligatable 3′ end or ligatable 5′ end to the 5′ end or the 3′ end of the circularizable probe, respectively, to generate a circular probe using the target nucleic acid as template; and d) amplifying the circular probe using rolling circle amplification (RCA) to generate an RCA product; and e) detecting the RCA product in the biological sample. In some embodiments, the cleaving in step b) is performed prior to the ligating in step c).

In some embodiments, the interrogatory region is in the stem-loop structure or a region on the 3′ or the 5′ of the stem-loop structure, and the interrogatory region is not in the first or second hybridization region. In some embodiments, the interrogatory region is in the stem of the stem-loop structure, the loop of the stem-loop structure, or a single-stranded region between the stem-loop structure and the first or second hybridization region. In some embodiments, the region of interest is between the first and second target sequences.

In some embodiments, the interrogatory region is in the first hybridization region and the region of interest is in the first target sequence, and the stem-loop structure blocks the circularization of the circularizable probe until the stem-loop structure or portion thereof is released.

In some embodiments, the interrogatory region is in the second hybridization region and the region of interest is in the second target sequence, and the stem-loop structure blocks the circularization of the circularizable probe until the stem-loop structure or portion thereof is released.

In any of the embodiments herein, the ligating step can comprise ligation selected from the group consisting of enzymatic ligation, chemical ligation, template-dependent ligation, and/or template independent ligation. In some embodiments, the enzymatic ligation may comprise using a ligase having an RNA-templated DNA ligase activity and/or an RNA-templated RNA ligase activity. In some embodiments, the enzymatic ligation may comprise using a ligase selected from the group consisting of a Chlorella virus DNA ligase (PBCV DNA ligase), a T4 RNA ligase, a T4 DNA ligase, and a single-stranded DNA (ssDNA) ligase.

In any of the embodiments herein, the method further comprises prior to the ligating step, a step of removing molecules of the probe or probe set that may not be bound to the target nucleic acid from the biological sample. In some embodiments, the method can further comprise prior to the ligating step, a step of removing molecules of the probe or probe set that may be bound to the target nucleic acid but may comprise one or more mismatches in the interrogatory region from the biological sample, and/or allowing the molecules (or portions thereof, e.g., the hybridization region that may comprise the interrogatory region) that may comprise one or more mismatches to dissociate from the target nucleic acid while the molecules that may comprise no mismatch in the interrogatory region remain bound to the target nucleic acid. In some embodiments, under the same conditions, the molecules that may comprise one or more mismatches may be less stably bound to the target nucleic acid than the molecules that may comprise no mismatch in the interrogatory region. In some embodiments, the method may comprise one or more stringency washes.

In any of the embodiments herein, the method may further comprise generating the product of the circularized probe in situ in the biological sample. In some embodiments, the product may be generated using rolling circle amplification (RCA). In some embodiments, the RCA may comprise a linear RCA, a branched RCA, a dendritic RCA, or any combination thereof. In any of the embodiments herein, the product can be generated using a polymerase selected from the group that may consist of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof.

Also disclosed herein in one aspect is a kit comprising a probe or probe set, and the probe or probe set comprises: i) a first hybridization region capable of hybridizing to a first target sequence in a target nucleic acid, ii) a second hybridization region capable of hybridizing to a second target sequence in the target nucleic acid, and iii) a duplex region, wherein upon hybridization of the probe or probe set to the target nucleic acid, the duplex region is positioned between the first and second hybridization regions. The first hybridization region, second hybridization region, and duplex region may be provided in one, two, three, or more probe molecules in any suitable combination.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

The following drawings illustrate certain embodiments of the features and advantages of this disclosure. These embodiments are not intended to limit the scope of the appended claims in any manner.

FIG. 1A depicts an exemplary circularizable probe comprising a duplex region within a stem-loop structure at an end of the probe hybridized to a target nucleic acid (for example, mRNA).

FIG. 1B depicts an exemplary first and second probe hybridized to a target sample, wherein the first probe comprises a duplex region at an end of the first probe.

FIG. 1C depicts an exemplary method comprising (1) nuclease cleavage of a duplex region from an end of a circularizable probe or probe set, wherein (2) release of the duplex or portion thereof generates a ligatable end of the probe or probe set, and (3) ligation of the ends of the probe or probe set generates a ligated probe.

FIGS. 2A-2C depict examples of a probe or probe set comprising a duplex region at different positions within the probe or probe set.

FIG. 3 depicts an exemplary probe comprising a stem-loop structure contacted with a biological sample comprising a target nucleic acid (for example, mRNA) comprising a region of interest such as a single nucleotide polymorphism (SNP) of interest.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Methods for detecting target nucleic acid using the target nucleic acid as a template for the ligation of a probe or probe sets, are available. For example, circularizable probe or probe set (e.g., padlock probes) may be used to hybridize to and detect a target nucleic acid. Padlock probes are linear nucleic acid molecules that comprise sequences complementary to the target nucleic acid at their 5′ and 3′ ends. Upon hybridization of a padlock probe to its target nucleic acid, the 5′ and 3′ ends are brought in close proximity to enable ligation. Padlock probe-based detection methods typically utilize DNA as a target and DNA padlock probes. Alternatively, RNA-template dependent ligation (e.g., RNA templated ligation or RTL) of DNA probes such as DNA padlock probes have also been demonstrated to be useful in detection of specific mRNAs. However, the RNA templated ligation (RTL) of DNA probes are not as efficient as DNA templated ligation of DNA probes and suffer from highly variable reaction efficiencies and poor specificities. Therefore, if the target nucleic acid is an mRNA, cDNA synthesis may be required to convert the target mRNA into cDNA in order to allow hybridization with the DNA padlock probe. In some instances, cDNA synthesis can lower the sensitivity of padlock reactions in situ and therefore may not be desirable. Furthermore, majority of ligase enzymes do not tolerate RNA templates as well and suffer from poor end-joining fidelity. The lack of fidelity of the ligase can lead to formation of a ligation product even when the padlock probe does not accurately match the sequence on the target mRNA, producing false positive results.

In situ methods for detecting mutations or single nucleotide polymorphisms (SNPs) also suffer from similar problems. For instance, padlock-RCA based SNP detection can use a ligase such as a T4 DNA ligase to ligate the padlock probe into a circular molecule, which will only occur when there has been specific base-pairing between the probe and the SNP of interest in the target nucleic acid. However, currently available designs for padlock-based SNP detection combined with RCA suffer from similar poor performance on RNA.

For RNA templated ligation reactions, the specificity of ligation is a very critical parameter for accurate RNA sequencing downstream. Current efforts using ribonucleotide bases at the 3′ end of the DNA can be expensive and may have challenges in manufacturability. Additionally, ribonucleotides at the 3′ ends do not prevent ligation of single-stranded nucleic acids, which can potentially further reduce specificity. Thus, there is a need for ligation methods with higher specificities for detection of target RNA molecules during RNA templated ligation reactions.

The present disclosure provides methods and compositions for improving ligation specificity for various applications. In some aspects, provided herein are modified probes or probe sets for improving ligation specificity. In some embodiments, the probe or probe set is a circularizable probe or probe set (e.g., a modified padlock probe). In some embodiments, the probe or probe set comprises a first probe and a second probe that can be ligated to form a ligated linear probe. In some embodiments, a 3′ or 5′ end of a probe (e.g., a circularizable probe, or one of a first and second probe) comprises a duplex region, wherein upon hybridization to the target nucleic acid, the duplex region is positioned between a first end and second end of the probe or probe set. For example, the duplex region can be positioned between the first end and second end of a circularizable probe. In another embodiment, the first end is an end of a first probe and the second end is an end of a second probe (e.g., the 3′ end of the first probe and the 5′ end of the second probe, or vice versa). In other embodiments, a 5′ or 3′ end of the probe or one or more probes in the probe set can comprise a non-ligatable blocking moiety or modification such as a dideoxynucleotide, 3′ hydroxyl group, or 5′ dephosphorylation that is removed by nuclease cleavage to generate a ligatable end. In other embodiments, the first and second end of the probe or probe set do not comprise a non-ligatable blocking moiety or modification such as a dideoxynucleotide, 3′ hydroxyl group, or 5′ dephosphorylation. In some embodiments, the duplex region itself (e.g., the hybridization between the two strands of the duplex) prevents ligation of the ends prior to cleavage of the duplex, without requiring a blocking moiety.

In some embodiments, a probe (e.g., a circularizable probe, or one of a first and second probe in a probe set) may comprise a 3′ region that does not hybridize to the target nucleic acid, ending with a dideoxynucleotide (e.g., ddNTP) at the 3′ end. In some embodiments, the 3′ region comprises a duplex region. In other embodiments, the 3′ region can be a single stranded flap. In some embodiments, the single-stranded flap is about 2-3 nucleotides in length and ends with a dideoxynucleotide. In some embodiments, a 3′ single stranded region can be combined with a 5′ duplex region in the probe or another probe in the probe set. In other embodiments, the probe or probe set comprises the 3′ single stranded region and does not comprise a 5′ duplex region. In some embodiments, digestion of the 3′ single stranded region by an exonuclease generates a ligatable 3′ end of the probe.

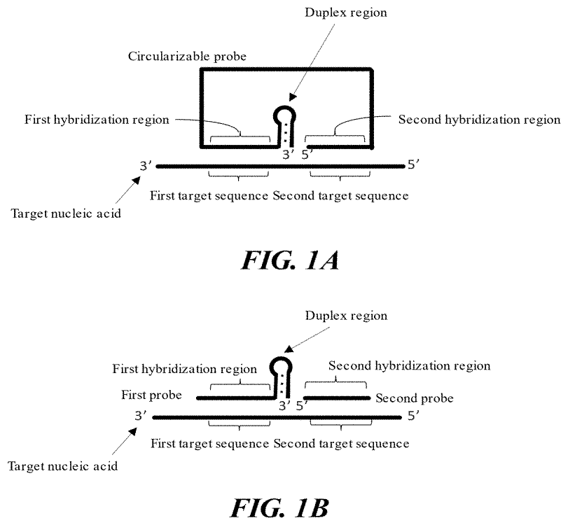

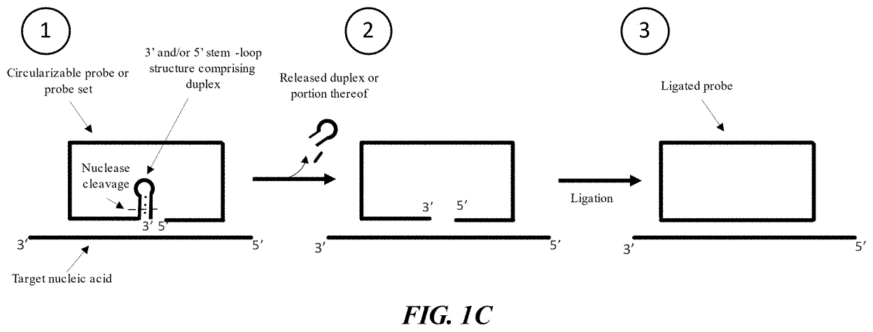

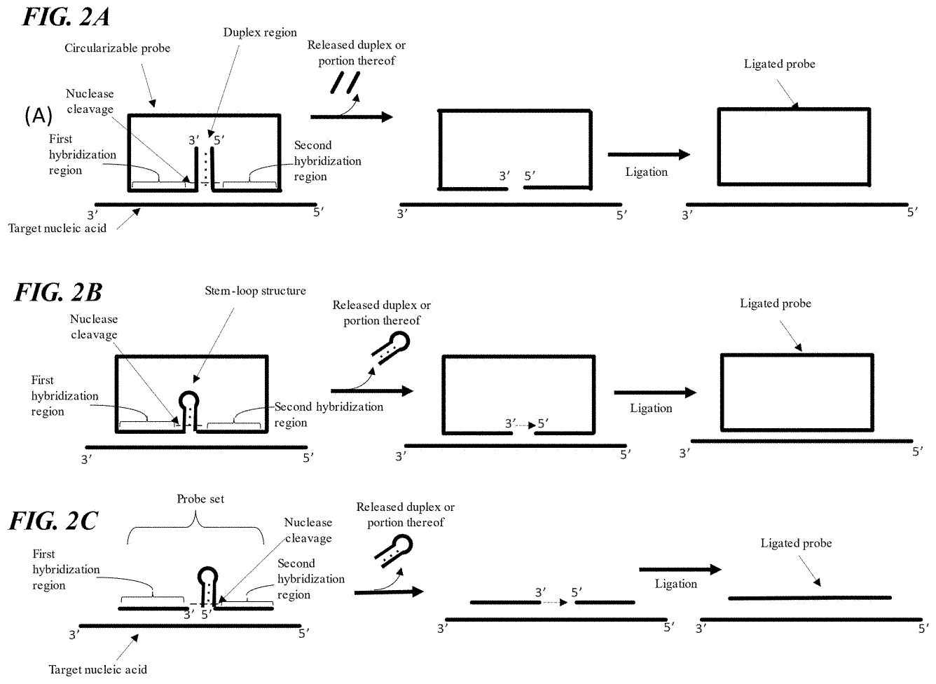

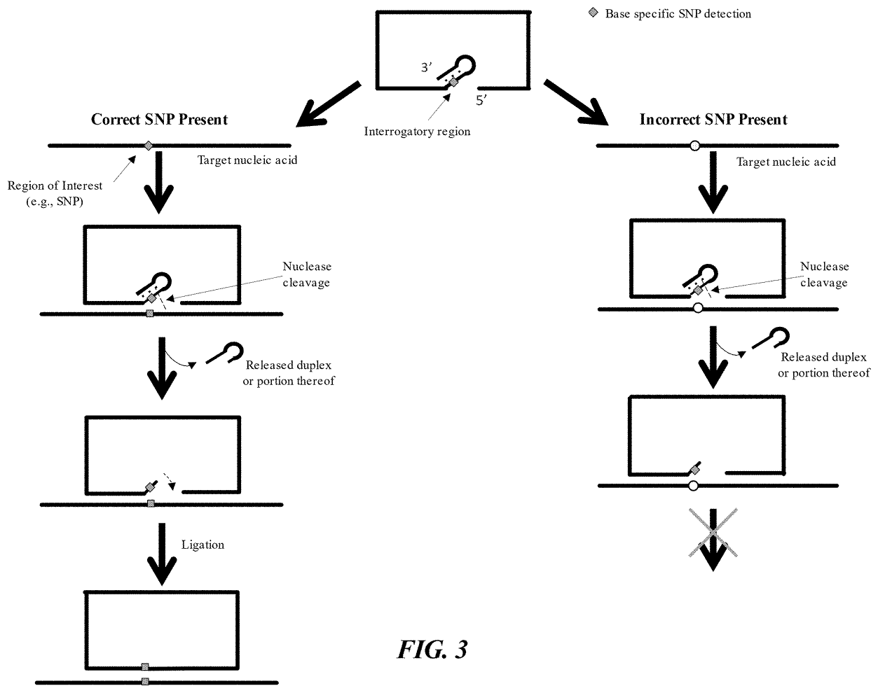

In some embodiments, the circularizable probe may comprise a stem-loop structure comprising a restriction site in the duplex region of the stem-loop ending with a 3′ dideoxynucleotide (e.g., ddNTP). In some embodiments, the stem-loop structure may comprise a duplex region. In some embodiments, the stem-loop structure may be a hairpin, comprising a duplex region. As shown in FIG. 1A, the duplex region (for example, within the stem-loop structure) may be at the 3′end and/or 5′end of a probe, such as a circularizable probe or one or a first and second probe in a probe set. In some embodiments, the duplex region is not within a stem-loop structure. In some embodiments, the duplex region may comprise a first end and a second end of a probe or probe set, as shown in FIG. 2A (e.g., a first and second end a circularizable probe, as shown in FIG. 2A). Similarly, the duplex region could comprise a first strand from a first probe in a probe set, and a second strand from a second probe in a probe set. For example, the duplex region could comprise the 3′ end of a first probe in a probe set, and a 5′ end of a second probe in the probe set, wherein cleaving the probe or probe set with a nuclease allows the 3′ end of the first probe to be ligated to the 5′ end of the second probe. In some embodiments, the duplex region and/or the stem-loop structure may be on one of the two probes of a given probe set, as shown in FIG. 2C.

In some embodiments, the method may comprise hybridizing a circularizable probe or probe set disclosed herein, to a target mRNA molecule in the biological sample. In some embodiments, the hybridized circularizable probe or probe set comprises a 3′ flap (e.g., 1, 2, 3, 4, 5 or more nucleotides that do not hybridize to the target nucleic acid) comprising a 3′ dideoxynucleotide. In some embodiments, the hybridized circularizable probe or probe set with a 3′ flap ending with a dideoxynucleotide at the 3′ terminus is circularized by ligating in the presence of an exonuclease. In some embodiments, only the exonuclease processed probes will be ligatable (e.g., capable of being ligated), thereby improving the specificity of the reaction.

In some embodiments, the method may comprise hybridizing a circularizable probe or probe set disclosed herein to a target mRNA molecule in the biological sample. In some embodiments, the circularizable probe or probe set comprises a duplex region at an end of the probe, wherein the duplex region is positioned between the first probe and the second probe when the probes are hybridized to the target nucleic acid. In some embodiments, the duplex structure is a stem-loop structure. In some embodiments, one of the two hybridized linear probes comprises a 3′ stem-loop structure ending with a 3′ dideoxynucleotide. In some embodiments, the hybridized circularizable probes with a 3′ stem-loop structure ending with a 3′ dideoxynucleotide are circularized by ligating in the presence of a restriction enzyme. In some embodiments, only the restriction enzyme processed probes will be ligatable, thereby improving the specificity of the reaction.

In some embodiments, the method may comprise hybridizing a linear probe set, disclosed herein, to a target mRNA molecule in the biological sample. In some embodiments, one of the two hybridized linear probes comprises a 3′ flap (e.g., 1, 2, 3, 4, 5 or more nucleotides that do not hybridize to the target nucleic acid) comprising a 3′ dideoxynucleotide. In some embodiments, one of the two hybridized linear probes with a 3′ flap ending with a 3′ dideoxynucleotide is ligated to the other probe of the probe set in the presence of an exonuclease(s). The RNA templated DNA ligation is performed in the presence of an exonuclease. In some embodiments, only the exonuclease processed probes will be ligatable, thereby improving the specificity of the reaction.

In some embodiments, the method may comprise hybridizing a linear probe set, disclosed herein, to a target mRNA molecule in the biological sample. In some embodiments, one of the two hybridized linear probes comprises a duplex region at an end of the probe, wherein the duplex region is positioned between the first probe and the second probe when the probes are hybridized to the target nucleic acid. In some embodiments, the duplex structure is a stem-loop structure. In some embodiments, one of the two hybridized linear probes comprises a 3′ stem-loop structure ending with a 3′ dideoxynucleotide. In some embodiments, one of the two hybridized linear probes with a 3′ stem-loop structure ending with a 3′ dideoxynucleotide, is ligated to the other probe of the probe set in the presence of restriction enzymes. The RNA templated DNA ligation is performed in the presence of a restriction enzyme(s). In some embodiments, only the restriction enzyme processed probes will be ligatable, thereby improving the specificity of the reaction.

Provided herein are circularizable probes for in situ applications and/or DNA probes for single cell analysis and spatial analysis applications. By redesigning the probes, ligation stringency can be specified by the additional requirement of either an exonuclease or a restriction enzyme to make the probes ligatable after hybridization. This will ensure that only hybridized probes that have been processed by either an exonuclease or a restriction enzyme will be ligatable. In some embodiments, only probes that are hybridized and processed to be capable of ligation can be used for downstream processing and be detected.

The present disclosure also provides methods and compositions for analysis of regions of interest in a target nucleic acid, such as a single nucleotide of interest (for example, SNPs or point mutations). In some embodiments, the circularizable probes provided herein can comprise an interrogatory region to detect SNPs in the target RNA molecule. In some embodiments, the interrogatory region is in the duplex region within the stem-loop structure (as shown in FIG. 3). In some embodiments, only hybridized probes that have been processed by either an exonuclease or a restriction enzyme will be ligatable, thereby improving the specificity of precisely detecting a single nucleotide polymorphism or a point mutation. Also provided are probes, sets of probes, compositions, kits, systems, and devices for use in accordance with the provided methods.

In some aspects, the provided methods and systems can be applied to detect, image, quantitate, or determine the presence or absence of one or more target nucleic acid(s) or portions thereof. In some aspects, the provided methods can be applied to detect, image, quantitate, or determine the sequence of one or more target nucleic acid(s), comprising sequence variants such as point mutations and SNPs.

In some aspects, the provided embodiments can be employed for in situ detection and/or sequencing of a target nucleic acid in a cell, e.g., in cells of a biological sample or a sample derived from a biological sample, such as a tissue section on a solid support, such as on a transparent slide.

In some aspects, the provided embodiments can be employed for and/or sequencing of a target nucleic acid in a single dissociated cell, e.g., in a cell suspension of a biological sample or a sample derived from a biological sample.

In some aspects, provided herein are in situ assays using microscopy as a readout, e.g., nucleic acid sequencing, hybridization, or other detection or determination methods involving an optical readout. In some aspects, detection or determination of a sequence of one, two, three, four, five, or more nucleotides of a target nucleic acid is performed in situ in a cell in an intact tissue. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product). In some embodiments, a method for spatially profiling analytes such as the transcriptome or a subset thereof in a biological sample is provided. Methods, compositions, kits, devices, and systems for these in situ assays, spatial genomics and transcriptomics assays, are provided. In some embodiments, a provided method is quantitative and preserves the spatial information within a tissue sample without physically isolating cells or using homogenates.

In some embodiments, through the use of various probe designs (e.g., various circularizable probes and/or linear probe designs such as described in Section III), the present disclosure provides methods for high-throughput profiling one or more single nucleotides of interest in a large number of targets in situ, such as transcripts and/or DNA loci, for detecting and/or quantifying nucleic acids in cells, tissues, organs or organisms.

In some aspects, the methods disclosed herein involve the use of one or more probes or probe sets that hybridize to a target nucleic acid, such as an RNA molecule, wherein at least one probe comprises a duplex region, wherein upon hybridization of the probe or probe set to the target nucleic acid, the duplex region is positioned between the first and second hybridization regions. In some embodiments, the probe or probe set includes a stem-loop structure that contains the duplex region. The specific probe or probe set design can vary. In some embodiments, the stem-loop structure or the duplex region in the primary probe (e.g., a DNA probe that directly binds to an RNA target) undergoes cleavage with a nuclease to generate ligatable ends. Exemplary nucleases include exonucleases, restriction endonucleases, Type IIS endonucleases, uracil-specific excision reagent enzymes, or nickases. In some embodiments, the methods disclosed herein are ligation dependent and involve only those probes that have been processed by a nuclease to undergo ligation. In some embodiments, the ligatable probes can be ligated, for example, either the ends of the probe can be ligated or two separate probes can be ligated together. In some embodiments, a primary probe (e.g., a DNA probe that directly binds to an RNA target) is amplified through rolling circle amplification.

In some embodiments, the provided methods involve analyzing, e.g., detecting or determining, one or more sequences present in a probe or probe set and/or in an amplification or extension product of a ligated probe or probe set, such as in an amplification product of a circularized probe. In some embodiments, the analysis comprises determining the sequence of all or a portion of the cleaved duplex region (for example the stem-loop structure). In some embodiments, the analysis can be used to correlate a sequence detected in an amplification product to a circularizable probe or probe set or a first and/or second probe used to form a ligated probe (e.g., via a cleaved stem-loop structure). In some embodiments, the detection of a sequence in an amplification product can provide information regarding the location and/or identity of an associated target nucleic acid (e.g., hybridized by the circularizable probe or first and second probe) in a sample. In some embodiments, due to amplification of one or more polynucleotides (e.g., a circularized probe or probe set), particular sequences present in the amplification product can be detected even when the template (e.g., the circularized probe or probe set) is present at low levels before the amplification.

In some aspects, the provided methods can be applied for various applications, such as for in situ analysis, comprising in situ detection (e.g., based on hybridization such as sequential hybridization) and/or sequencing of target nucleic acids and multiplexed nucleic acid analysis. In some aspects, the provided methods can be for in situ detection and/or identification of a region (e.g., single nucleotide) of interest in target nucleic acids. In some aspects, the provided methods can be used with a spatial array.

In some embodiments, provided herein are methods for analyzing a biological sample comprising a plurality of target nucleic acid molecules (for example, RNA) comprising: a) contacting the biological sample with a probe or probe set, wherein the probe or probe set comprises: i) a first hybridization region capable of hybridizing to a first target sequence in a target nucleic acid in the biological sample, ii) a second hybridization region capable of hybridizing to a second target sequence in the target nucleic acid, and iii) a duplex region, wherein upon hybridization of the probe or probe set to the target nucleic acid, the duplex region is positioned between the first and second hybridization regions; b) cleaving the probe or probe set with a nuclease to generate a first ligatable end and releasing the duplex region or a portion thereof; c) ligating the first ligatable end to a second ligatable end in the probe or probe set to generate a ligated probe; and d) detecting the ligated probe or a product thereof in the biological sample. In some embodiments, for example, as shown in FIG. 1A, FIG. 1C, FIG. 2A and FIG. 2B, the duplex region and/or the stem-loop structure is included in the same molecule. In some embodiments, for example, as shown in FIG. 1B and FIG. 2C, the probe set comprising a duplex region in the stem-loop structure comprises at least two different molecules. In some embodiments, a site in the duplex region is cleaved in step b). In some embodiments, the cleaving in step b) generates the first and/or second ligatable end. In some embodiments, the method further comprises detecting the released duplex region or portion thereof and/or a product thereof. In some embodiments, a cleaved portion (or portions) of the probe(s), such as a cleaved duplex region or portion thereof, is washed from the target RNA molecule, under conditions in which molecule(s) of the nuclease processed probe remain hybridized to the target RNA molecule in biological sample.

In some embodiments, provided herein are methods for analyzing a biological sample using RNA templated ligation, comprising: a) contacting the target RNA with a first probe and a second probe simultaneously or in any order, wherein: the first probe comprises: i) a first hybridization region capable of hybridizing to a first target RNA sequence in the target RNA, and ii) a stem-loop structure the second probe comprises a second hybridization region capable of hybridizing to a second target RNA sequence in the target RNA, and upon hybridization of the first and second probes to the target RNA, the stem-loop structure is at positioned between the first and second hybridization regions; b) cleaving the first probe with a nuclease to generate a first ligatable end, thereby releasing the stem-loop structure or a portion thereof; c) ligating the first ligatable end to a second ligatable end in the second probe to generate a ligated probe using the target RNA as template; and d) detecting the ligated probe or a product thereof. In some embodiments, RNA templated ligation comprises RNA-template dependent ligation of DNA probes.

In some embodiments, provided herein are methods for analyzing a biological sample using rolling circle amplification (RCA) comprising: a) contacting the biological sample with a circularizable probe comprising: i) a first hybridization region capable of hybridizing to a first target sequence in a target nucleic acid in the biological sample, ii) a second hybridization region capable of hybridizing to a second target sequence in the target nucleic acid, and iii) a stem-loop structure at the 3′ or 5′ end of the circularizable probe, wherein upon hybridization of the circularizable probe to the target nucleic acid, the stem-loop structure is positioned between the first and second hybridization regions; b) cleaving the circularizable probe with a nuclease to generate a ligatable 3′ end or ligatable 5′ end and release the stem-loop structure or a portion thereof; c) ligating the ligatable 3′ end or ligatable 5′ end to the 5′ end or the 3′ end of the circularizable probe, respectively, to generate a circular probe using the target nucleic acid as template; d) amplifying the circular probe using rolling circle amplification (RCA) to generate an RCA product; and e) detecting the RCA product in the biological sample.

In some embodiments, e.g., as shown in FIG. 3, provided herein are methods for analyzing a biological sample comprising a plurality of target molecules (e.g., RNA) comprising a single nucleotide of interest, the methods comprising: a) contacting the biological sample with a circularizable probe comprising: i) a first hybridization region capable of hybridizing to a first target sequence in a target nucleic acid in the biological sample, ii) a second hybridization region capable of hybridizing to a second target sequence in the target nucleic acid, iii) a stem-loop structure at the 3′ or 5′ end of the circularizable probe, wherein the target nucleic acid comprises a region of interest and the stem-loop structure comprises an interrogatory region, and wherein upon hybridization of the circularizable probe to the target nucleic acid, the stem-loop structure is positioned between the first and second hybridization regions; b) cleaving the circularizable probe with a nuclease to generate a ligatable 3′ end or ligatable 5′ end and releasing the stem-loop structure or a portion thereof, thereby allowing the interrogatory region to hybridize to the region of interest; c) if the interrogatory region is complementary to the region of interest, ligating the ligatable 3′ end or ligatable 5′ end to the 5′ end or the 3′ end of the circularizable probe, respectively, to generate a circular probe using the target nucleic acid as template; and d) amplifying the circular probe using rolling circle amplification (RCA) to generate an RCA product; and e) detecting the RCA product in the biological sample.

In some embodiments, the target nucleic acid comprises a region of interest and the probe or probe set comprises an interrogatory region. In some embodiments, the interrogatory region is in the duplex region and is not capable of hybridizing to the region of interest until the cleaving in step b).

In some embodiments, the cleaving in step b) and the ligating in step c) are performed simultaneously. In some embodiments, the cleaving in step b) is performed prior to the ligating in step c). In some embodiments, the method further comprises prior to the ligating step, a step of removing molecules of the probe or probe set that are bound to the target nucleic acid but comprise one or more mismatches in the interrogatory region from the biological sample, and/or allowing the molecules (or portions thereof, e.g., the hybridization region comprising the interrogatory region) comprising one or more mismatches to dissociate from the target nucleic acid while the molecules comprising no mismatch in the interrogatory region remain bound to the target nucleic acid.

In some embodiments, provided herein are methods for assessing one or more target nucleic acids, such as a plurality of different mRNAs, in a biological sample, such as a cell or a tissue sample (such as a tissue section). In some embodiments, the target nucleic acid comprises RNA. In some embodiments, the target nucleic acid is an mRNA. In some embodiments, wherein the probe or probe set comprises DNA.

In some aspects, the provided methods are employed for in situ analysis of target nucleic acids, for example for in situ sequencing or multiplexed analysis in intact tissues or a sample with preserved cellular or tissue structure. In some aspects, the provided methods can be used to detect or determine the identity or amount or frequency in situ of single nucleotides of interest in target nucleic acids, for instance of single nucleotide polymorphisms of genes of interest.

In some aspects, the provided methods comprise one or more steps of ligating the polynucleotides, for instance of ligating the ends of a circularizable probe to form a circularized probe and/or ligating a first probe and second probe to form a ligated linear probe product. In some aspects, the provided methods involve a step of amplifying one of the polynucleotides (e.g., a circularized probe produced therefrom), to generate an amplification product. In some aspects, the provided methods involve a step of detecting and/or determining the sequence of all or a portion of the amplification product. In some aspects, the provided methods involve performing one or more of the steps described herein, simultaneously and/or sequentially.

Particulars of the steps of the methods can be carried out as described herein, for example in Sections III-VIII; and/or using any suitable processes and methods for carrying out the particular steps.

### II. Samples, Analytes, and Target Sequences

A. Samples

A sample disclosed herein can be or derived from any biological sample. Methods and compositions disclosed herein may be used for analyzing a biological sample, which may be obtained from a subject using any one of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a single cell sample, such as a dissociated single cell sample. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any one of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or circularizable probe or probe set (e.g., a padlock probe), or a first and second probe. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable probe or probe set (e.g., a padlock probe) or to generate a ligated first-second probe.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any one of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample maybe utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of analytes out of the sample, and/or to facilitate transfer of species (such as probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some aspects, when two or more analytes are analyzed, a first and second probe (e.g., a probe set of a first and second linear probe) that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity using any one of a variety of methods (e.g., streptavidin beads).

Alternatively, one or more species of RNA can be down-selected (e.g., removed) using any one of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V.A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g., an affinity binding partner) can be developed. Such a specific binding partner may be a nucleic acid probe (for a nucleic acid analyte) and may lead directly to the generation of an RCA template (e.g., a circularizable probe). Alternatively, the specific binding partner may be coupled to a nucleic acid, which may be detected using an RCA strategy, e.g., in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g., including but not limited to protein-DNA complexes, which may or may not be covalently bound to one another, and which may be the same or different. Thus, in addition to cells or microorganisms, such a complex analyte may also be a protein complex or protein interaction. Such a complex or interaction may thus be a homo- or hetero-multimer. Aggregates of molecules, e.g., proteins may also be target analytes, for example aggregates of the same protein or different proteins. The analyte may also be a complex between proteins or peptides and nucleic acid molecules such as DNA or RNA, e.g., interactions between proteins and nucleic acids, e.g., regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

(ii) Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any one of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any one of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer binding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

C. Target Sequences

In some embodiments, provided herein are methods and compositions for analyzing endogenous analytes (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed. A target sequence for a probe disclosed herein may be comprised in any analyte disclose herein, including an endogenous analyte (e.g., a viral or cellular nucleic acid), a labelling agent, or a product of an endogenous analyte and/or a labelling agent.

### III. Polynucleotides and Hybridization Complexes

Disclosed herein in some aspects are nucleic acid probes and/or probe sets that are introduced into a cell or used to otherwise contact a biological sample such as a tissue sample or a sample comprising isolated cells. In some aspects, the nucleic acid probes and/or probe sets comprise at least two hybridization regions capable of hybridizing to target sequences in the target nucleic acid. In some aspects, the target nucleic acid comprises RNA and the probe or probe set comprises DNA and/or RNA. In some aspects, the probe or probe set comprise a first hybridization region capable of hybridizing to a first target sequence in a target nucleic acid and a second hybridization region capable of hybridizing to a second target sequence in a target nucleic acid in the biological sample. In some aspects, the probe or probe sets comprise an interrogatory region, and the target nucleic acid comprises a region of interest. The probe or probe sets comprise a duplex region (such as a stem-loop structure) wherein upon hybridization of the probe or probe set to the target nucleic acid, the duplex is positioned between the first and second hybridization regions. In some aspects, the probe or probe set is cleaved with a nuclease to generate a first ligatable end. In some aspects, the design of the probe or probe set ensures that only those probes that are hybridized to the target RNA and are processed by the exonuclease or restriction enzyme will be ligatable. This allows for improved ligation specificity and stringency. Compared to a probe design that includes incorporation of ribonucleotide bases at the 3′ end of the DNA probe, which may be expensive and be challenging to manufacture, provided herein are alternative designs and methods for improving ligation specificity.

The probes may comprise any one of a variety of entities that can hybridize to a nucleic acid, typically by Watson-Crick base pairing, such as DNA, RNA, LNA, PNA, etc., depending on the application. The nucleic acid probe(s) typically contains a hybridization region that is able to bind to at least a portion of a target nucleic acid, in some embodiments specifically. The nucleic acid probe may be able to bind to a specific target nucleic acid (e.g., an mRNA, or other nucleic acids as discussed herein). In some embodiments, the nucleic acid probes may be detected using a detectable label, and/or by using secondary nucleic acid probes able to bind to the nucleic acid probes. In some embodiments, the nucleic acid probes are compatible with one or more biological and/or chemical reactions. For instance, a nucleic acid probe disclosed herein can serve as a template or primer for a polymerase, a template or substrate for a ligase, a substrate for a click chemistry reaction, and/or a substrate for a nuclease (e.g., endonuclease for cleavage).

Provided herein are methods involving the use of one or more probes (e.g., a circularizable probe such as a padlock probe) for analyzing one or more target nucleic acid(s), such as a target nucleic acid (for example, a messenger RNA) present in a cell or a biological sample, such as a tissue sample. Also provided are probes, sets of probes, compositions, kits, systems and devices for use in accordance with the provided methods. In some aspects, the provided methods and systems can be applied to detect, image, quantitate, or determine the presence or absence of one or more target nucleic acid(s) or portions thereof (e.g., presence or absence of sequence variants such as point mutations and SNPs). In some aspects, the provided methods can be applied to detect, image, quantitate, or determine the sequence of one or more target nucleic acid(s), comprising sequence variants such as point mutations and SNPs.

In some aspects, a target nucleic acid disclosed herein comprises any polynucleotide nucleic acid molecule (e.g., DNA molecule; RNA molecule, modified nucleic acid, etc.) for assessment in accordance with the provided embodiments, such as a polynucleotide present in a cell. In some embodiments, the target nucleic acid is a coding RNA (e.g., mRNA). The target may, in some embodiments, be a single RNA molecule. In other embodiments, the target may be at least one RNA molecule, e.g., a group of 2, 3, 4, 5, 6 or more RNA molecules. These RNA molecules may differ in molecule type, and/or may differ in sequence. In some embodiments, the target nucleic acid is, for example, a non-coding RNA (e.g., tRNA, rRNA, microRNA (miRNA), mature miRNA or immature miRNA). In some embodiments, the target nucleic acid is a splice variant of an RNA molecule (e.g., mRNA, pre-mRNA, etc.) in the context of a cell. A suitable target nucleic acid can therefore be an unspliced RNA (e.g., pre-mRNA, mRNA), a partially spliced RNA, or a fully spliced RNA, etc. Target nucleic acids of interest may be variably expressed, e.g., have a differing abundance, within a cell population, wherein the methods of the present disclosure allow profiling and comparison of the expression levels of nucleic acids, comprising but not limited to, RNA transcripts, in individual cells. A target nucleic acid can also be a DNA molecule, e.g., a denatured genomic, viral, plasmid, etc. For example, the methods can be used to detect copy number variants, e.g., in a cancer cell population in which a target nucleic acid is present at different abundance in the genome of cells in the population; a virus-infected cells to determine the virus load and kinetics, and the like.

In some aspects, the methods provided herein are used to analyze a target nucleic acid, e.g., a messenger RNA molecule. In some embodiments, the target nucleic acid is an endogenous nucleic acid present in a biological sample. In some embodiments, the target nucleic acid is present in a cell in a tissue, for instance in a tissue sample or tissue section. In some embodiments, the tissue sample is an intact tissue sample or a non-homogenized tissue sample. In some embodiments, the tissue sample is a fresh tissue sample. In some embodiments, the tissue has previously been processed, e.g., fixed, embedded, frozen, or permeabilized using any one of the steps and/or protocols described in Section II. In some embodiments, the target nucleic acid is an exogenous nucleic acid contacted with a biological sample. In some embodiments, the target nucleic acid is present in a single cell, for instance in a dissociated single cell sample.

In some aspects, the provided embodiments can be employed for in situ detection and/or sequencing of a target nucleic acid in a cell, e.g., in cells of a biological sample or a sample derived from a biological sample, such as a tissue section on a solid support, such as on a transparent slide.

In some aspects, the methods disclosed herein involve the use of one or more probes or probe sets that hybridize to a target nucleic acid, such as an RNA molecule, wherein at least one probe is a circularizable probe comprising a first hybridization region and a second hybridization region that hybridize to a target nucleic acid. The specific probe or probe set design can vary. In some embodiments, the probes or probe sets (e.g., circularizable probe or a pair of linear probes) may comprise a duplex region. For example, as shown in FIG. 1A, the duplex region (for example, within the stem-loop structure) may be at the 3′end of the circularizable probe. In some instances, the duplex region (for example, within the stem-loop structure) may be at the 5′end of the circularizable probe. In some embodiments, the duplex region may be formed as shown in FIG. 2A, comprising two strands, one strand from each end of the circularizable probe. In some embodiments, the duplex region and/or the stem-loop structure may be on one of the two probes of a given linear probe set, as shown in FIG. 1B. In some embodiments, the duplex region may be formed from two separate strands, one strand from each of the two probes of the pair of linear probes (e.g., 3′ end of a first probe and 5′ end of a second probe).

FIGS. 1A and 1C depict an exemplary circularizable probe comprising a duplex region within a stem-loop structure on the 3′ end of the probe. Alternatively, the circularizable probe may comprise a duplex region within a stem-loop structure on the 5′ end of the probe. FIG. 1B depicts an exemplary probe set comprising a duplex region within a stem-loop structure on the 3′ end of the first probe. In some instances, the probe set may alternatively comprise a duplex region within a stem-loop structure on the 5′ end of the second probe. The probe(s) is/are contacted with a target nucleic acid (for example, RNA). The probe(s) does not contain ligatable 5′ or 3′ end due to the presence of the stem-loop structure at the 5′ or 3′ end. The probe can comprise a 5′ phosphate group and a 3′ terminal residue that is blocked from ligation. Alternatively, the probe can comprise a 5′ phosphate group and a 3′ hydroxyl group which cannot be ligated to the 5′ phosphate group due to the presence of the stem-loop structure at the 3′ end of the probe. The probe(s) can comprise two hybridization regions complementary to regions on the target nucleic acid (e.g., first target sequence and second target sequence). Upon hybridization, the stem-loop structure is positioned between the two hybridization regions of the probe(s). Upon nuclease-mediated cleavage of the stem-loop structure at a site within or outside of the stem-loop structure, the stem-loop structure or a portion can be released. The nuclease-mediated cleavage can generate ligatable 5′ and/or 3′ ends. For instance, as shown the 3′ stem-loop structure can be cleaved to generate a ligatable 3′ end. In other examples (not shown), a 5′ stem-loop structure can be cleaved to generate a ligatable 5′ end, or the probe can comprise stem-loop structures on both ends which are cleaved to generate ligatable 5′ and 3′ ends. The ligatable 5′ and 3′ ends can optionally be extended by a polymerase, using the RNA target nucleic acid as a template and ligated using a ligase (such as a T4 DNA ligase, a PB CV-1 DNA ligase, or a T4 RNA ligase 2 (T4 Rnl2)). The processed probe can be ligated to circularize the circularizable probe. The ligated probe can then be amplified to a rolling circle amplification product (RCA) and then detected. In some instances, the processed probe set comprising the linear first and second probes can be ligated together to form a linear ligated probe product (e.g., ligated first-second probe).

FIG. 2 depicts examples of a probe or probe set comprising a duplex region at different positions within the probe or probe set. The probe in FIG. 2A comprises a duplex region that is not part of a stem-loop structure. The duplex region comprises two strands, one on each of the 5′ and 3′ ends. The two strands are complementary to each other thereby forming a duplex region. Nuclease-mediated cleavage of one or both the strands of the duplex region may release the duplex region and generate two ligatable ends that can be ligated to form a circularized product. The probe in FIG. 2B comprises a duplex region within a stem-loop structure. Nuclease-mediated cleavage can release the stem-loop structure and generate two ligatable ends that can be ligated to form a circularized product. The linear probe set in FIG. 2C comprises two linear probes hybridized to two adjacent complementary sequences on the target nucleic acid. One of the two probes of the probe set comprises a duplex region within the stem-loop structure. Nuclease-mediated cleavage (for instance, at both strands of the stem-loop structure) can release the stem-loop structure and generate ligatable ends that can be ligated to form a linear ligated probe (e.g., ligated first-second probe).

In some embodiments, a circularized probe or probe set (e.g., a DNA probe that directly binds to an RNA target) is amplified through rolling circle amplification. In some embodiments a circularizable probe or probe set is ligated, circularized, and amplified. In some embodiments the circularizable probe or probe set is ligated using a target nucleic acid as a template, optionally wherein the target nucleic acid is an RNA. In some embodiments, a circularizable probe or probe set can be generated by cleaving a stem-loop structure in a circular probe (e.g., as shown in FIG. 2B). In some embodiments, the circular probe or circularizable probe or a probe set may contain one or more barcodes. In some embodiments, one or more barcodes are indicative of a sequence in the target nucleic acid (e.g., a sequence of a region of interest such as a single nucleotide of interest (e.g., SNPs or point mutations), a dinucleotide sequence, or a short sequence of about 5 nucleotides in length).

In some aspects, the provided methods involve analyzing, e.g., detecting or determining, one or more sequences present in the polynucleotides and/or in an amplification product, such as in an amplification product of a circularized probe, which may comprise one or more barcode sequences. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region (e.g., a single nucleotide) of interest in a target nucleic acid. In some embodiments, the analysis can be used to correlate a sequence detected in an amplification product to a circularizable probe (e.g., via a barcode). In some embodiments, the detection of a sequence in an amplification product can provide information regarding the location and/or identity of an associated target nucleic acid (e.g., hybridized by the probe(s)) in a sample. In some embodiments, due to amplification of one or more polynucleotides (e.g., a circularizable probe), particular sequences present in the amplification product or complementary sequences thereof can be detected even when a polynucleotide is present at low levels before the amplification. For example, the number of copies of the barcode sequence(s) and/or a complementary sequence thereof is increased by virtue of the amplification of a probe comprising the barcode sequence(s) and/or complementary sequence thereof, thereby enabling specific and sensitive detection of a signal indicative of the identity of a short region (e.g., a single nucleotide) of interest in a target nucleic acid. In particular embodiments, the amplification product is an in situ rolling circle amplification (RCA) product of a circularized probe.

In some embodiments, provided herein are methods for assessing one or more target nucleic acids, such as a plurality of different mRNAs, in a biological sample, such as a cell or a tissue sample (such as a tissue section). In some embodiments, the target nucleic acid comprises DNA. In some embodiments, the target nucleic acid comprises RNA. In some embodiments, the target nucleic acid comprises mRNA. In some embodiments, the probe comprises DNA. In some embodiments, the target nucleic acid is RNA and the probe comprises DNA.

In some aspects, the provided methods are employed for in situ analysis of target nucleic acids, for example for in situ sequencing or multiplexed analysis in intact tissues or a sample with preserved cellular or tissue structure. In some aspects, the provided methods can be used to detect or determine the identity or amount in situ of single nucleotides of interest in target nucleic acids, for instance of single nucleotide polymorphisms of genes of interest. In some aspects, the provided methods are employed for analysis of target nucleic acids using a spatial array, as discussed, for example in Section VI. In some aspects, the provided methods are employed for analysis of target nucleic acids in a dissociated single cell, as discussed, for example in Section VII.

In some aspects, the provided methods involve a step of contacting, or hybridizing, one or more polynucleotides, such as any one of the probes described herein, to a cell or a sample containing a target nucleic acid with a region (e.g., single nucleotide) of interest in order to form a ligatable hybridization complex. In some aspects, the provided methods comprise cleaving the duplex (such as a stem-loop structure) to generate ligatable ends or ligatable ends. In some aspects, the provided methods comprise one or more steps of ligating the ligatable or ligatable ends of polynucleotides, for instance of ligating the ligatable ends of a circularizable probe to form a circularized probe. In some aspects, the one or more steps of ligating the ligatable or ligatable ends of polynucleotides, comprise ligating the ligatable ends of a probe set to form a linear probe. In some aspects, the provided methods involve a step of amplifying one of the polynucleotides (e.g., a padlock probe, a circularized probe or a linear probe produced therefrom), to generate an amplification product. In some aspects, the provided methods involve a step of detecting and/or determining the sequence of all or a portion of the amplification product (for example, of one or more barcodes contained in the amplification product) and/or one or more of the polynucleotides with or without amplification, for instance any barcodes contained therein. In some aspects, the provided methods involve performing one or more of the steps described herein, simultaneously and/or sequentially.

In some aspects, the methods provided herein comprise use of one or more polynucleotides, e.g., probe or probes that comprise i) a first hybridization region that hybridizes to a first target sequence in the target nucleic acid, ii) a second hybridization region that hybridizes to a second target sequence in the target nucleic acid, and iii) a duplex region. In some aspects, upon hybridization of the probe or probe set to the target nucleic acid, the duplex region is positioned between the first and second hybridization regions. In some aspects, the duplex region is 3′ or 5′ to the first hybridization region or is 5′ to 3′ to the second hybridization region. In some aspects, the duplex region is between or at a 3′ end or at a 5′ end of the probe or a probe in the probe set. In some embodiments, the duplex region is an internal region of the probe or a probe in the probe set. In some aspects, the duplex region is at the 3′ end and 5′ end of the probe or probe in the probe set.

In some embodiments, the duplex region comprises a first and a second strand. In some embodiments, the two strands may be part of the same probe comprising self-complementary strands. In some embodiments, the first strand is at the 5′ end and the second strand is at the 3′ end of the probe molecule or vice versa. In some embodiments, the two strands are part of two different probes (for instance, one strand is a part of a first probe and the other strand is part of a second probe). In some embodiments, the first strand is at the 5′ end of the first probe and the second strand is at the 3′ end of the second probe. In some embodiments, the first strand is at the 3′ end of the first probe and the second strand is at the 5′ end of the second probe. In some embodiments, the first strand and the second strand are continuous and are at the 5′ end or at the 3′ end of the probe molecule. In some embodiments, the probe set comprises a first probe and a second probe, and the first strand and the second strand are continuous. In some embodiments, the first strand and the second strand are at the 5′ end or at the 3′ end of the first probe. In some embodiments, the first strand and the second strand are at the 5′ end or at the 3′ end of the second probe.

In some embodiments, the probe or probe set comprises a stem-loop structure comprising the duplex region wherein the duplex region is in the stem of the stem-loop structure. In some aspects, the duplex region is at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 base pairs in length. In some aspects, the duplex region is at least 5 nucleotides in length. In some instances, the duplex region can include a functional sequence. The functional sequence can be a primer sequence. The primer sequence can be used to amplify the duplex region or the stem-loop structure before cleavage, contemporaneously with cleavage, or after cleavage of the duplex region from the nucleic acid probe. In some aspects, instead of detecting the presence of oligonucleotides (e.g., circularizable probes or probe sets) that hybridize directly to a target nucleic acid, the methods disclosed herein include detecting the cleavage of a non-hybridized sequence of an oligonucleotide (for example, a duplex region) and further analyzing to determine the presence of absence of an analyte (for example, an mRNA molecule) of interest. In some aspects, the method includes determining all or part of the duplex region. In some aspects, the duplex region further includes a barcode sequence. When the duplex is cleaved, the duplex can be used to identify a hybridization and/or ligation event where the circularizable probe or the first and second nucleic acid probe undergo ligation. In some aspects, the probe or a probe set does not comprise a duplex region. In some aspects, the probe or probe set comprises an additional sequence 5′ to a target-specific binding site which is not hybridized to the target nucleic acid molecule. In some aspects, upon hybridization of the probe to the target nucleic acid molecule the additional sequence forms a 5′ flap containing one or more nucleotides at its 3′ end that is cleaved prior to ligation. The 5′ flap is not part of a duplex. In some embodiments, a probe or probe set comprises a duplex at the 3′ end and a 5′ flap at the 5′ end.

In some embodiments, a circularizable probe herein comprises from the 3′ to 5′ direction: a duplex region, a first hybridization region, a barcode, and a second hybridization region (e.g., as shown in FIG. 1A). In some embodiments, a circularizable probe herein comprises from the 5′ to 3′ direction: a duplex region, a second hybridization region, a barcode, and a first hybridization region. In some embodiments, a circularizable probe comprises from the 3′ to 5′ direction: a first strand of a duplex region, a first hybridization region, a barcode, a second hybridization region, and a second strand of the duplex region (e.g., as shown in FIG. 2A). In some embodiments, a circularizable probe herein comprises from the 5′ to 3′ direction: a first strand of a duplex region, a first hybridization region, a barcode, a second hybridization region, and a second strand of the duplex region.

In some embodiments, a probe set comprises from the 3′ to 5′ direction of a first probe: a duplex region and a first hybridization region, and a second probe from 5′ to 3′ direction: a second hybridization region and optionally barcodes on overhangs. In some embodiments, a probe set comprises from the 3′ to 5′ direction of a first probe: a first hybridization region and a second probe from 5′ to 3′ direction: a duplex region, and a second hybridization region, with optional barcodes on overhangs. In some embodiments, a probe set comprises from 3′ to 5′ direction of a first probe: a first strand of a duplex region and a first hybridization region, and a second probe from 5′ to 3′ direction: a second strand of a duplex region, a second hybridization region, and optional barcodes on overhangs.

In some embodiments, there is provided a first probe or probe set (e.g., a first circularizable probe or probe set or a first linear probe or probe set) for detecting a first region of interest (e.g., a first nucleotide of interest such as a SNP) and a second probe (e.g., a second circularizable probe or probe set or a second linear probe or probe set) for detecting an alternate region of interest (e.g., an alternate SNP). For example, a region of interest may comprise one of two variants, wherein the first nucleotide of interest is C, and the alternate nucleotide of interest is A. However, the methods and compositions provided herein can discriminate between any two nucleotides. For example, the first probe or probe set can comprise a first barcode sequence and a first interrogatory nucleotide (e.g., G) capable of basepairing with a first nucleotide of interest (e.g., C), and the second probe or probe set can comprise a second barcode sequence, and an alternate interrogatory nucleotide (e.g., T) capable of basepairing with an alternate nucleotide of interest (e.g., A). Binding of the interrogatory region to the region of interest (e.g., a SNP) allows ligation of probe(s) (e.g., ligation of the ends of the circularizable probe or probe set to form a circularized probe, or ligation of the first probe and second probe to form a ligated linear probe). In some embodiments, the concentration of each of the provided probes or probe sets contacted with the sample can be tuned for sensitivity and selectivity.

In some embodiments, provided herein is a method for analyzing the sequence of a region of interest in a target nucleic acid molecule comprising a probe or probe set disclosed herein, wherein the probe or probe set hybridized to the target nucleic acid molecule comprises a 5′ hybridization region and a 3′ hybridization region, and cleavage of a duplex region allows ligation of the 5′ hybridization region and the 3′ hybridization region. In some embodiments, the 5′ hybridization region and/or the 3′ hybridization region comprises an interrogatory region for analyzing the sequence of a region of interest in the first or second region, respectively. In some embodiments, the shorter one of the 5′ hybridization region and the 3′ hybridization region comprises the interrogatory region, such as an interrogatory nucleotide for a single nucleotide of interest in the target nucleic acid. In some embodiments, the sequence of interest in a first molecule of the target nucleic acid is complementary to the interrogatory region, whereas the sequence of interest in a second molecule of the target nucleic acid comprises a mismatch with the interrogatory region. In some embodiments, the mismatch is not at the 5′ or 3′ terminal nucleotide of the circularizable probe. In some embodiments, the method further comprises cleaving the duplex region at 3′ or 5′ end of the probe to generate ligatable ends. In some embodiments, the method further comprises ligating the 5′ and the 3′ hybridization regions of the circularizable probe hybridized to the first molecule of the target nucleic acid to form a circularized probe, whereas under the same or similar conditions the 5′ and the 3′ hybridization regions of the circularizable probe hybridized to the second molecule of the target nucleic acid are not ligated. In some embodiments, the method further comprises detecting the circularized probe in the biological sample. In some embodiments, a signal associated with the circularized probe is detected in situ at the location of the first molecule of the target nucleic acid. In some embodiments, a probe may comprise a 5′ hybridization region and a 3′ hybridization region each comprising an interrogatory region for detecting a different nucleotide of interest (e.g., a SNP).

In some embodiments, provided herein is a method for analyzing a biological sample, comprising contacting the biological sample with a circularizable probe or probe set. In some embodiments, the circularizable probe or probe set comprises a linear oligonucleotide sequence that, upon hybridization to a target nucleic acid, such as an RNA molecule, forms a probe that can be circularized. The probe can be circularized via ligation (or primer extension followed by ligation) using the target RNA and/or the DNA splint as a template. In any one of the embodiments herein, the probe may be adapted for various different probe ligation and detection methods.

In some aspects, the probe comprises an interrogatory region that may or may not be complementary to a region of interest in the target nucleic acid. In some aspects, the interrogatory region is or comprises the first nucleotide positioned at the point connecting the duplex region to a hybridization region of the probe or probe set. In some embodiments, the interrogatory region is at a 3′ or 5′ ligatable end. In some embodiments, the interrogatory region is a single nucleotide (an interrogatory nucleotide). In some embodiments, the interrogatory region can be internal, that is, not at the point where the duplex connects to a hybridization region of the probe or probe set. In some embodiments, the interrogatory region is located within the first or second hybridization region. In some embodiments, the interrogatory region is no more than about 5, 4, 3, 2, or 1 nucleotides away from a ligatable end (e.g., a 3′ hydroxy or a 5′ phosphate group) of the first or second hybridization region (optionally wherein the ligatable end is generated by cleavage with a nuclease as described herein). In some aspects, the interrogatory region is in the duplex region or in the stem-loop structure. In some aspects, the interrogatory region is in the stem and/or the loop of the stem-loop structure. In some aspects, the interrogatory region is positioned between the 3′ or 5′ end of a hybridization region and the nuclease (e.g., the endonuclease or nickase) recognition or cleavage site. For example, FIG. 3 depicts an interrogatory region located within the duplex region, between the 3′ or 5′ end of a hybridization region and the nuclease cleavage site. After cleavage of one or both strands of the duplex region, the cleaved duplex region may no longer be stable, allowing the cleaved portion of the duplex region to be removed (e.g., in a wash step). The interrogatory region is then free to hybridize to the complementary region of interest in the target nucleic acid, allowing ligation of the first and second ligatable ends of the probe or probe set. In some embodiments, the interrogatory region in the duplex region or stem-loop structure is not capable of hybridizing to the region of interest until the cleavage step (e.g., cleaving of the duplex region by an endonuclease described herein). In some aspects, if the interrogatory region on the circularizable probe is complementary to the region of interest (for example a SNP) in the target nucleic acid, the interrogatory region hybridizes to the region of interest in the target nucleic acid. In some aspects, if the interrogatory region on the circularizable probe is not complementary to the region of interest (for example a SNP) in the target nucleic acid, the interrogatory region does not hybridize to the region of interest in the target nucleic acid. Upon cleavage of the duplex region and generation of ligatable end(s), the hybridized interrogatory region is ligated to the adjacent ligatable end of the probe or probe set to form a circularized probe or a ligated linear probe. If there is a mismatch between the interrogatory region and the region of interest in the target nucleic acid, a partial hybridization prevents ligation from proceeding.

In any one of the embodiments herein, the interrogatory region may comprise a variety of different locations on the probe. For example, the interrogatory region may comprise, e.g., the 2nd, 3rd, 4th, 5th, 6th, 7th, 8th, 9th, and/or 10th positions from the ligation site on the probe hybridization region and/or either or both ends of the probe, though it is not limited to any one of those particular positions. In some embodiments, the interrogatory region is no more than about 5, 4, 3, 2, or 1 nucleotides away from a ligatable end of the probe or probe set. In some embodiments, the interrogatory region comprises a nucleotide at a ligatable end of the probe or probe set. In some embodiments, the interrogatory region comprises a portion of the first ligatable end and a portion of the second ligatable end.

In any one of the embodiments herein, the target nucleic acid may be, e.g, an mRNA molecule. In any one of the embodiments herein, the interrogatory region can comprise a single interrogatory nucleotide for detecting, e.g., a particular SNP in the target nucleic acid. In any one of the embodiments herein, the interrogatory region can comprise more than one interrogatory nucleotide.

In any one of the embodiments herein, a circularizable probe or probe set can comprise one, two, three, four, or more ribonucleotides. In some embodiments, a circularizable probe disclosed herein can comprise one, two, three, four, or more ribonucleotides in a DNA backbone. In any one of the embodiments herein, the one or more ribonucleotides can be at and/or near a ligatable 3′ end of the circularizable probe or probe set. The circularizable probe may comprise an optional 3′ RNA base. The presence of a complementary base in the SNP location in the target nucleic acid favors stable hybridization of the circularizable probe and subsequent probe circularization. In contrast, the interrogatory nucleotide-containing arm is less stably hybridized when there is a mismatch between the interrogatory nucleotide and the SNP location in the target nucleic acid. The interrogatory nucleotide-containing arm can dissociate from the target nucleic acid and prevent circularization of the probe by a ligase having poor fidelity (e.g., on RNA templates), thus reducing false positive signals due to indiscriminating ligase activity. In some embodiments, the circularizable probe or probe set can comprise a uracil residue positioned between the duplex region and a hybridization region of the probe or probe set, linking the duplex region and the hybridization region. In some embodiments, the uracil residue is positioned between the duplex region and the interrogatory region. In some embodiments, the method comprises contacting the biological sample with a nuclease, such as a uracil-specific excision reagent enzyme to excise the uracil residue, thereby cleaving the duplex region and generating a ligatable end of the probe or probe set.

In any one of the embodiments herein, a linear probe set (e.g., first and second probes) can comprise one, two, three, four, or more ribonucleotides. In some embodiments, a probe set disclosed herein can comprise one, two, three, four, or more ribonucleotides in a DNA backbone. In any one of the embodiments herein, the one or more ribonucleotides can be at and/or near a ligatable 3′ end of a probe (e.g., first probe) of the probe set. The probe may comprise an optional 3′ RNA base. The presence of a complementary base in the SNP location in the target nucleic acid favors stable hybridization of the probe of the probe set. In contrast, the interrogatory nucleotide-containing probe (e.g., first probe) of the probe set is less stably hybridized when there is a mismatch between the interrogatory nucleotide and the SNP location in the target nucleic acid. The interrogatory nucleotide-containing probe can dissociate from the target nucleic acid and prevent ligation of the probes in the probe set by a ligase having poor fidelity (e.g., on RNA templates), thus reducing false positive signals due to indiscriminating ligase activity. In some embodiments, the linear probe or probe set can comprise a uracil residue positioned between the duplex region and a hybridization region of the probe or probe set, linking the duplex region and the hybridization region. In some embodiments, the uracil residue is positioned between the duplex region and the interrogatory region. In some embodiments, the method comprises contacting the biological sample with a nuclease, such as a uracil-specific excision reagent enzyme to excise the uracil residue, thereby cleaving the duplex region and generating a ligatable end of the linear probe or probe set.

FIG. 3 depicts an exemplary probe comprising a stem-loop structure. The probe is contacted with a biological sample comprising a target nucleic acid (for example, RNA) comprising a region of interest such as a single nucleotide polymorphism (SNP) of interest. The probe can comprise a 5′ phosphate group and a stem-loop structure on its 3′ end. The hybridization region comprises an interrogatory region (for example, an interrogatory nucleotide) that is complementary to the region of interest (for example, correct SNP present, left side of FIG. 3). Alternatively, the probe may comprise an interrogatory region (for example, an interrogatory nucleotide) that is not complementary to the region of interest (for example, incorrect SNP present, right side of FIG. 3). The probe is processed by a nuclease to release the stem-loop structure and generate ligatable ends. If the target nucleic acid comprises the SNP of interest such that it is complementary to the interrogatory nucleotide, hybridization is stable and the 3′ and 5′ ends can be ligated to each other (left side of FIG. 3). However, if the target nucleic acid does not comprise a SNP complementary to the interrogatory nucleotide, hybridization is unstable, and the 3′ and 5′ ends are not ligated (right side of FIG. 3). In some embodiments, the interrogatory region can comprise one or more nucleotides that are part of the recognition sequence of the nuclease. In some embodiments, the stem-loop structure can be cleaved regardless of the identity of the interrogatory nucleotide. For example, cleavage of the duplex region can be constant across all probes or probe sets, regardless of the identity of the interrogatory region. In some embodiments, a probe comprising a stem-loop structure comprising a probe comprising an interrogatory region that is not complementary to the region of interest can be cleaved (e.g., as shown in the example of FIG. 3, right panel, depicting a incorrect SNP present in the target nucleic acid), but unstable hybridization between the interrogatory region and the region of interest (e.g., the single nucleotide of interest) will prevent subsequent ligation. Although FIG. 3 depicts a circularizable probe, it will be understood that an interrogatory region in a linear probe or probe set could similarly discriminate between different sequences of a region of interest, generating a ligated linear probe only in the presence of the correct sequence of the region of interest (e.g., the correct SNP).

### IV. Enzymatic Cleavage

In some embodiments, the methods provided herein comprise cleaving the probe or probe set with a nuclease to generate one or more ligatable ends and releasing the duplex region or a portion thereof. In some embodiments, the method comprises cleaving the probe or probe set within the duplex region (e.g., a site in the duplex region), for example, using a nuclease (such as a restriction enzyme or nickase) that recognizes a double stranded recognition site within the duplex. In some aspects, the probe or probe set described herein, comprises a non-ligatable end and the cleaving step removes the non-ligatable end. In some aspects, the design of the double-stranded duplex region by itself or the double-stranded duplex region in the stem of a stem-loop structure ensures that only those probes that are hybridized to the target nucleic acid and are cleaved by the nuclease will be ligatable. In some embodiments, the probe or probe set comprises a single-stranded region linking the duplex region to the first or second hybridization region, and a site in the single-stranded region is cleaved.

In some embodiments, the method comprises contacting the biological sample with a nuclease, such as a restriction endonuclease. In some embodiments, the sample is contacted with at least any of 1 U, 2 U, 5 U, 10 U, 20 U, 30 U, 40 U, 50 U, 60 U, 70 U, 80 U, 90 U, or 100 U of a restriction endonuclease. One unit of restriction endonuclease activity is defined as the amount of enzyme (measured in units, U) that will cleave 1 μg of DNA (usually lambda DNA) to completion in 1 hour at the optimum temperature for the enzyme, usually 37° C. In some embodiments, the restriction endonuclease cleaves at a recognition sequence that is 4, 5, 6, 7, 8, or more base pairs in length. In some embodiments, the restriction endonuclease cleaves in the recognition sequence and/or at a site outside of the recognition sequence.

Suitable restriction endonucleases include restriction endonucleases that generate blunt ends or sticky ends. Exemplary restriction endonucleases include, but is not limited to, AanI, Acc16I, AccII, AccBSI, AcvI, AfaI, AfeI, AjiI, AluBI, Aor51HI, BalI, BmgBI, Bsh1235I, BsnI, Bsp68I, BspANI, BspFNI, BsrBI, BssNAI, Bst11071, BstSNI, BsuRI, BtuMI, DinI, DraI, Ecl136II, Eco105I, Eco147I, Eco321, Eco47III, Eco53kI, Eco721, EcoICRI, EcoRV, EgeI, EheI, FspI, HpaI, HpyCH4V, KspAI, MbiI, MluNI, Mox20I, MscI, Msp20I, MssI, MvnI, NaeI, NruI, NsbI, PceI, PdiI, PmaCI, PmeI, PmII, PsiI, PspCI, PvuII, RruI, ScaI, SfoI, SmaI, SnaBI, SrfI, SseBI, SspI, StuI, SwaI, ZraI, ZrmI, AatII, AbsI, Acc65I, AccIII, AcII, AciI, AfIII, AgeI, AhII, Alw44I, Aor13HI, ApaI, ApaLI, AscI, AseI, AsiGI, AsiSI, Asp718I, AspA2I, AspLEI, AsuII, AvrII, BamHI, BauI, BbvCI, BcII, BcuI, BfaI, BfrI, BgIII, BlnI, BmgT120I, BmtI, Bpu14I, Bsa29I, BseAI, BseCI, BsePI, BseX3I, BseYI, BshTI, BshVI, BsiWI, BspACI, Bsp119I, Bsp120I, Bsp13I, Bsp1407I, Bsp19I, BspDI, BspEI, BspHI, BspMAI, BspOI, BspT104I, BspTI, BsrGI, BssHII, BssMI, BssSI-v2, Bst2BI, BstAFI, BstAUI, BstBI, BstHHI, BstMBI, BstZI, Bsu15I, BsuTUI CciI, CciNI, CfoI, Cfr42I, Cfr9I, ClaI, CspAI, Csp6I, CviAII, CviQI, DpnII, EagI, EclXI, EcoRI, EcoT22I, FaeI, FatI, FauNDI, FbaI, FseI, GsaI, HapII, HhaI, Hin1II, Hin6I, HindIII, HpaI, HpaII, HpySE526I, Hsp92II, KasI, Kpn2I, KpnI, Ksp22I, KspI, Kzo9I, MaeI, MboI, MluCI, MauBI, MfeI, MluI, Mly113I, Mph1103I, MreI, MroI, MroNI, MspCI, MseI, MspI, MunI, NarI, NcoI, NdeI, NdeII, NgoMIV, NheI, NlaIII, NotI, Nsil, NspV, PaeI, PaeR7I, PagI, PalAI, PauI, PciI, Pfl23II, PinAI, Ple19I, PluTI, PscI, PshBI, Psp124BI, Psp1406I, PspFI, PspLI, PspOMI, PstI, PteI, PvuI, RgaI, RigI, RsaNI, SacI, SacII, SaII, SaqAI, Sau3AI, SbfI, SdaI, SfaAI, Sfr274I, Sfr202I, SfuI, SgfI, SgrBI, SgrDI, SgsI, SlaI, SpeI, SphI, Sse83871, Sse9I, SsiI, SspDI, SspMI, SstI, TaiI, TaqI, TaqI-v2, TasI, TrulI, TspMI, Vha464I, VneI, VspI, XbaI, XhoI, XmaI, or Zsp2I. In some embodiments, the restriction endonuclease is EcoRV.

In some embodiments, the restriction endonuclease is capable of recognizing double-stranded recognition sequences with variable or degenerate sequence base positions. Examples of restriction endonucleases recognizing double-stranded recognition sequences with variable sequence base positions include AasI, Acc36I, AccB7I, AcIWI, AdeI, AfiI, AhdI, Alw26I, AlwI, AlwNI, Asp700I, AspS9I, AxyI, BbsI, BccI, BcoDI, BfuAI, BgII, BlpI, Bme1390I, BmeRI, BmiI, BmrFI, BmrI, BmuI, BoxI, BpiI, Bpu10I, Bpu1102I, BsaBI, BsaJI, Bsc4I, Bse21I, Bse8I, BseDI, BseGI, BseJI, BseLI, BseMI, BsII, BsmAI, BsmBI-v2, BsmI, Bso31I, Bsp1720I, BspLI, BspMI, BspPI, BspQI, BspTNI, BsrDI, BsrI, BssECI, Bst4CI, Bst6I, BstAPI, BstC8I, BstEII, BstENI, BstF5I, BstMAI, BstMWI, BstPAI, BstPI, BstV2I, BstXI, Bsu36I, BtsCI, Btsl-v2, BtsIMutI, BveI, Cac8I, CaiI, Cfr13I, DdeI, DraIII, DrdI, DriI, DseDI, Eam1104I, Eam1105I, EarI, Eco31I, Eco81I, Eco91I, EcoNI, EcoO65I, Esp3I, FauI, Fnu4HI, Fsp4HI, HinfI, Hpyl66II, Hpyl88I, Hpyl88III, Hpy8I, HpyF10VI, HpyF3I, LguI, LmnI, MaeIII, MroXI, MsII, NIaIV, OliI, PaqCI, PciSI, PctI, PdmI, PflFI, PflMI, PfoI, PleI, PpsI, PshAI, PspEI, PspN4I, PspPI, PstNI, PsyI, RseI, SapI, ScrFI, SfiI, SmiMI, TaaI, Tth111I, Van91I, XagI, XcmI, and XmnI.

In some embodiments, the restriction endonuclease cleaves in the recognition sequence and/or at a site outside of the recognition sequence. Exemplary restriction endonucleases include, but is not limited to, AcuI, AlwI, BaeI, BbsI, BbsI-HF, BbvI, BccI, BceAI, BcgI, BciVI, BcoDI, BfuAI, BmrL, BpmL, BpuEI, Bsal-HFv2, BsaXI, BseRI, Bsgl, BsmAI, BsmBI-v2, BsmFI, BsmI, BspCNI, BspMI, BspQI, BsrDI, Bsrl, BtgZI, BtsCI, Btsl-v2, BtsIMutl, CspCI, EasI, EciI, Esp3I, FauI, FokI, Hgal, HpyAV, MboII, MIyI, MnII, NmeAIII, PaqCI, PIeI, SapI, and SfaNI. The restriction endonucleases may generate blunt ends or sticky ends.

In some embodiments, cleaving the double-stranded recognition sequence comprises incubating the sample with the nuclease (e.g, a restriction endonuclease). In some embodiments, the method comprises incubating the sample with the nuclease (e.g., a restriction endonuclease) for at least 20 minutes, at least 30 minutes, at least 35 minutes, at least 40 minutes, at least 45 minutes, at least 50 minutes, at least 60 minutes, at least 80 minutes, at least 100 minutes, or at least 120 minutes. In some embodiments, the incubation with the nuclease is performed for 20-60 minutes, 20-45 minutes, 20-120 minutes, 30-120 minutes, 30-60 minutes, or 30-90 minutes. In some embodiments, the incubation with the restriction endonuclease is performed at 30° C.-40° C., e.g., at 37° C.

In some embodiments, method comprises contacting the biological sample with a nuclease, such as a Uracil-Specific Excision Reagent (e.g., USER®) enzyme or enzymes. In some aspects, the duplex region is linked to the probe (e.g., linked to a hybridization region of the probe) by a uracil residue (e.g., the duplex can be linked to a hybridization region of the probe or probe set by a single-stranded region comprising a uracil residue). In some embodiments, a uracil-specific excision reagent can comprise a uracil DNA glycosylase (UDG), which catalyzes the excision of a uracil base, forming an abasic (apyrimidinic) site while leaving the phosphodiester backbone intact. The uracil-specific excision reagent can further comprise an Endonuclease VIII. The lyase activity of Endonuclease VIII breaks the phosphodiester backbone at the 3′ and 5′ sides of the abasic site so that base-free deoxyribose is released. In some embodiments, the uracil-specific excision reagent is an enzyme that combines the activities of a Uracil DNA glycosylase (UDG) and an endonuclease VIII (e.g., a USER® enzyme). In some embodiments, the uracil-specific excision reagent catalyzes the excision of the uracil residue generating a single nucleotide gap at the location of the residue. In some embodiments, the sample is contacted with at least any of 1 U, 2 U, 5 U, 10 U, 20 U, 30 U, 40 U, 50 U, 60 U, 70 U, 80 U, 90 U, or 100 U. One unit (U) is defined as the amount of enzyme required to nick 10 pmol of a 34 mer oligonucleotide duplex containing a single uracil base, in 15 minutes at 37° C. in a total reaction volume of 10 μl.

In some embodiments, method comprises contacting the biological sample with a nuclease, such as a nickase. Nickases or nicking endonucleases hydrolyze only one strand to produce DNA molecules that are nicked rather than cleaved. Like restriction endonucleases, nickases recognize short specific DNA sequences and cleave the DNA strand at a fixed position relative to the recognition sequence. In some embodiments, the nickase recognizes a specific DNA sequence of 1, 2, 3, 4, 5, 6, or more nucleotides in length. However, unlike restriction endonucleases, nickases cleave only one strand of a double stranded polynucleotide. A non-exhaustive list of examples of nickases includes: Nb.BbvCI, Nb.BsmI, Nb.BsrDI, Nb.BtsI, Nt.Alwl, Nt.BbvC, Nt.BpulOl, Nt.BstNBI, Nt.BspQI, Nt.BsmAI, Nt.BbvCI, and Nt.CviPII. The latter enzyme is commercially available from ThermoFisher Scientific; the others are available from e.g., New England Biolabs. The lower case letter “b” or “t” in the name of the nicking enzyme denotes whether the enzyme makes a nick in the bottom or top strand respectively (the accepted convention being that the top strand runs from free 5′ end on the left to free 3′ end on the right, with the bottom strand in the opposite orientation). In some embodiments, the nickase is Nt.BbvCI. In some embodiments, the nickase is Nt.CviPII. In some embodiments, the sample is contacted with at least any of 1 U, 2 U, 5 U, 10 U, 20 U, 30 U, 40 U, 50 U, 60 U, 70 U, 80 U, 90 U, or 100 U. One unit is defined as the amount of enzyme, for instance Nt.BstNBI, required to digest 1 μg T7 DNA in 1 hour at 55° C. in a total reaction volume of 50 μl.

In some embodiments, the nuclease (for example, a restriction endonuclease) is a rare cutter for endogenous sequences in the biological sample. Thus, the digestion of endogenous sequences such as chromosomal DNA can be minimized by selection of the restriction endonuclease. In some embodiments, the nuclease recognizes a sequence in a DNA/DNA duplex region in the probe or probe set. In some embodiments, the nuclease does not cleave DNA/RNA heteroduplexes. In some embodiments, the target nucleic acid is an RNA molecule, and a nuclease that does not cleave DNA/RNA heteroduplexes will not cleave the hybridization regions of the probe or probe sets hybridized to the RNA molecule. Thus, in some embodiments, the first and second hybridization regions are not designed to avoid including a sequence that could be recognized by the nuclease if it were present a DNA/DNA duplex. In other embodiments, the probe or probes in a probe set can be designed to avoid including additional recognition and/or cleavage sites (e.g., at locations other than the duplex region or region linking the duplex region to the rest of the probe or probe set.

In some embodiments, the probe or probe set described herein, comprises a non-ligatable end. In some instances, a non-ligatable 3′ end comprises a 3′ dideoxynucleotide (e.g., ddNTP). In some aspects, the non-ligatable end comprises a modified nucleotide. For example, a non-ligatable 3′ end may lack a 3′ hydroxyl group, or a non-ligatable 5′ end may be 5′ dephosphorylated. In some aspects, the cleaving step removes the non-ligatable end.

In some embodiments, a cleavage step is followed by a wash step to remove a cleaved stem-loop structure, (FIGS. 1C and 2A), the two strands of a duplex region (FIG. 2A), and/or any portion thereof (FIG. 2C), associated with the circularizable probe or probe set from the sample. In some embodiments, the wash step is performed using conditions that allow the probe or probe set to remain hybridized to the target nucleic acid. In some embodiments, the removal step comprises performing a stringent wash. In some aspects, the released duplex region or portion thereof and/or product thereof is detected outside the biological sample. In some aspects, the released duplex or portion thereof and/or product thereof is captured and/or detected on an array, and in some embodiments, it is analyzed by nucleic acid sequencing.

In some embodiments, a probe or probe set disclosed herein (e.g., a circularizable probe or probe set, or a first and second probe) can comprise a 3′ duplex region as described herein, and may further comprise a 5′ flap. The 5′ flap may be recognized by a structure-specific cleavage enzyme, e.g., an enzyme capable of recognizing the junction between the single-stranded 5′ overhang (flap) and the hybridized circularizable probe or probe set (e.g., padlock probe), and cleaving the single-stranded overhang. It will be understood that the branched three-strand structure which is the substrate for the structure-specific cleavage enzyme may be formed by 5′ end of one probe part and the 3′ end of another probe part when both have hybridized to the target nucleic acid molecule, as well as by the 5′ and 3′ ends of a one-part probe. Enzymes suitable for such cleavage include Flap endonucleases (FENS), which are a class of enzymes having endonucleolytic activity and being capable of catalyzing the hydrolytic cleavage of the phosphodiester bond at the junction of single- and double-stranded DNA. Thus, in some embodiments, cleavage of the additional sequence 5′ to the first target-specific binding site is performed by a structure-specific cleavage enzyme, e.g., a Flap endonuclease. Suitable Flap endonucleases are described in Ma et al. 2000. JBC 275, 24693-24700 and in US 2020/022424 and may include P. furiosus (Pfu), A. fulgidus (Afu), M. jannaschii (Mja) or M. thermoautotrophicum (Mth). In other embodiments an enzyme capable of recognizing and degrading a single-stranded oligonucleotide having a free 5′ end may be used to cleave an additional sequence (5′ flap) from a structure as described above. Thus, an enzyme having 5′ nuclease activity may be used to cleave a 5′ additional sequence. Such 5′ nuclease activity may be 5′ exonuclease and/or 5′ endonuclease activity. A 5′ nuclease enzyme is capable of recognizing a free 5′ end of a single-stranded oligonucleotide and degrading said single-stranded oligonucleotide. A 5′ exonuclease degrades a single-stranded oligonucleotide having a free 5′ end by degrading the oligonucleotide into constituent mononucleotides from its 5′ end. A 5′ endonuclease activity may cleave the 5′ flap sequence internally at one or more nucleotides. Further, a 5′ nuclease activity may take place by the enzyme traversing the single-stranded oligonucleotide to a region of duplex once it has recognized the free 5′ end, and cleaving the single-stranded region into larger constituent nucleotides (e.g., dinucleotides or trinucleotides), or cleaving the entire 5′ single-stranded region, e.g., as described in Lyamichev et al. 1999. PNAS 96, 6143-6148 for Taq DNA polymerase and the 5′ nuclease thereof. Preferred enzymes having 5′ nuclease activity include Exonuclease VIII, or a native or recombinant DNA polymerase enzyme from Thermus aquaticus (Taq), Thermus thermophilus or Thermus flavus, or the nuclease domain therefrom.

### V. Ligation and Amplification

In some aspects, after nuclease-driven cleavage of the duplex region and the generation of ligatable (e.g., ligatable) ends of the probe or probe sets described in Section IV, the assay further comprises one or more steps such as ligation, extension and/or amplification of the probe or probe set hybridized to the target nucleic acid. In some embodiments, the methods of the present disclosure include the step of performing rolling circle amplification in the presence of a target nucleic acid of interest.

In some embodiments, the method described herein releases the cleaved duplex from the probe or probe set thereby generating at least one ligatable end. In some embodiments, the stem-loop structure is part of the same oligonucleotide molecule. For example, the stem-loop structure connects a first hybridization region and a second hybridization of a circularizable probe as shown in FIG. 2B. In this instance, cleavage of the stem-loop structure generates two ligatable ends (for example, first and second ligatable ends of a circularizable probe or probe set). In another example, the stem-loop structure can connect a first and second hybridization region of a linear probe. In this case, cleavage of the stem-loop structure generates a first and a second probe comprising ligatable ends, wherein the ligatable ends can be ligated to form a ligated linear probe. In some aspects, both ligatable ends are part of the same oligonucleotide molecule (FIG. 2B). In some embodiments, the stem-loop structure is part of the different oligonucleotide molecules (for example, the stem-loop structure is one a first strand of a two part linear probe pair as shown in FIG. 2C). In some embodiments, the 3′ end and the 5′ end of the circularizable probe or probe set can be ligated using the target nucleic acid (e.g., RNA) as a template. In some embodiments, the first ligatable end is a 3′ ligatable end and the second ligatable end is a 5′ ligatable end. In some other embodiments, the first ligatable end is a 5′ ligatable end and the second ligatable end is a 3′ ligatable end. In some embodiments, a ligated probe is generated using the target nucleic acid as template. In some embodiments, the 3′ end and the 5′ end are ligated without gap filling prior to ligation. In some embodiments, the ligation of the 3′ end and the 5′ end is preceded by gap filling. The gap may be 1, 2, 3, 4, 5, or more nucleotides.

In some embodiments, the method comprises using a circular or circularizable construct hybridized to the target nucleic acid comprising the region of interest to generate a product (e.g., comprising a sequence of the region of interest associated with the target nucleic acid). In some aspects, the product is generated using RCA. In any one of the embodiments herein, the method can comprise ligating the ends of a circularizable probe hybridized to the target RNA to form a circularized probe. In any one of the embodiments herein, the method can further comprise generating a rolling circle amplification product of the circularized probe. In some embodiments, the RCA comprises a linear RCA. In some embodiments, the RCA comprises a branched RCA. In some embodiments, the RCA comprises a dendritic RCA. In some embodiments, the RCA comprises any combination of the foregoing. In any one of the embodiments herein, the method can further comprise detecting a signal associated with the rolling circle amplification product in the biological sample. In some embodiments, a ligation product of a first and second probe is generated. In some aspects, the ligation product or a derivative thereof (e.g., extension product) can be detected. In some cases, RCA is not performed.

In some embodiments, the circular construct is formed using ligation. In some embodiments, the circular construct is formed using template primer extension followed by ligation. In some embodiments, the ligated probe is generated using the target nucleic acid as template. In some embodiments, the circular construct is formed by providing an insert between ends to be ligated. In some embodiments, the circular construct is formed using a combination of any one of the foregoing. In some embodiments, the ligation is a DNA-DNA templated ligation. In some embodiments, the ligation is an RNA-RNA templated ligation. In some embodiments, the ligation is a RNA-DNA templated ligation, for instance ligation of DNA probes is dependent on the RNA template. In some embodiments, a splint is provided as a template for ligation.

In some embodiments, the circular construct is directly hybridized to the target nucleic acid. In some embodiments, the circular construct is formed from a modified circularizable probe or probe set (e.g., any of the circularizable probes or probe sets comprising a duplex region described herein). In some embodiments, the circular construct is formed from a probe or probe set capable of DNA-templated ligation. See, e.g., U.S. Pat. No. 8,551,710, which is hereby incorporated by reference in its entirety. In some embodiments, the circular construct is formed from a DNA probe or probe set capable of hybridizing to an RNA target nucleic acid (e.g., RNA-templated ligation). Exemplary RNA-templated ligation (RTL) probes and methods are described in US 2020/022424 which is incorporated herein by reference in its entirety.

The nature of the ligation reaction depends on the structural components of the polynucleotides used to form the circularizable probe. In some embodiments, the polynucleotides comprise complementary docking regions that post cleavage of the duplex, self-assemble the two or more polynucleotides into a circularizable probe, that is either ready for ligation because no gaps exist between the docking regions, or is ready for a fill-in process, which will then permit the ligation of the polynucleotides to form the circularizable probe. In some embodiments, the docking regions are complementary to a splint.

In some embodiments, a 3′ end and a 5′ end of the circularizable probe or probe set can be ligated using the target nucleic acid (e.g., RNA) as a template. In some embodiments, the 3′ end and the 5′ end are ligated without gap filling prior to ligation. In some embodiments, the ligation of the 3′ end and the 5′ end is preceded by gap filling. The gap may be 1, 2, 3, 4, 5, or more nucleotides.

In some embodiments, ligating the first ligatable end to a second ligatable end in the probe or probe set may comprise enzymatic ligation, chemical ligation, template dependent ligation, and/or template independent ligation. In some embodiments, ligating the first ligatable end to a second ligatable end in the probe or probe set is a template dependent ligation, for example, wherein the ligation depends on hybridization of an interrogatory region to a region of interest in the target nucleic acid. In any one of the embodiments herein, the ligation can comprise using a ligase having an RNA-templated DNA ligase activity and/or an RNA-templated RNA ligase activity. In some embodiments, the enzymatic ligation involves use of a ligase (e.g., an RNA ligase, a DNA ligase). Ligases comprise ATP-dependent double-strand polynucleotide ligases, NAD-i-dependent double-strand DNA or RNA ligases and single-strand polynucleotide ligases, for example any one of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In any one of the embodiments herein, the ligation can comprise using a ligase selected from the group consisting of a Chlorella virus DNA ligase (PBCV DNA ligase), a T4 RNA ligase, a T4 DNA ligase, and a single-stranded DNA (ssDNA) ligase. In any one of the embodiments herein, the ligation can comprise using a PBCV-1 DNA ligase or variant or derivative thereof and/or a T4 RNA ligase 2 (T4 Rnl2) or variant or derivative thereof. In some embodiments, the ligase is a T4 RNA ligase. In some embodiments, the ligase comprises a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. In some embodiments, a direct ligation occurs between ends of the polynucleotides hybridize immediately adjacently to one another to form a substrate for a ligase enzyme resulting in their ligation to each other (intramolecular ligation). In some embodiments, an indirect ligation is performed when the ends of the polynucleotides hybridize non-adjacently to one another, (for instance, separated by one or more intervening nucleotides or gaps). In some embodiments, said ends are not ligated directly to each other, but instead occurs either via the intermediacy of one or more intervening (so-called gap or gap-filling (oligo)nucleotides) or by the extension of the 3′ end of a probe to fill the “gap” corresponding to said intervening nucleotides (intermolecular ligation). In some cases, the gap of one or more nucleotides between the hybridized ends of the polynucleotides may be filled by one or more gap (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of hybridized mismatched ligation substrates (e.g., wherein the interrogatory region is not complementary to the region of interest, such that the probe or probes are expected to have a slightly lower Tₘ around the mismatch) over hybridized fully base-paired ligation substrates (e.g., wherein the interrogatory region is complementary to the region of interest). Thus, high-fidelity ligation can be achieved through a combination of the intrinsic selectivity of the ligase active site and balanced conditions to reduce the incidence of annealed mismatched dsDNA.

In some embodiments, the method can further comprise prior to ligating the first ligatable end to a second ligatable end in the probe or probe set, a step of removing molecules of the probe or probe set (for instance, the stem-loop structure or the duplex region) that are not bound to the target nucleic acid from the biological sample. In any one of the embodiments herein, the method can further comprise prior to the ligating step, a step of removing molecules of the probe or probe set that are bound to the target nucleic acid but comprise one or more mismatches in the interrogatory region from the biological sample. In any one of the embodiments herein, the method can further comprise prior to the ligating step, a step of allowing probe molecules that are bound to the target nucleic acid but comprise one or more mismatches in the interrogatory region to dissociate from the target nucleic acid, while probe molecules comprising no mismatch in the interrogatory region remain bound to the target nucleic acid. In any one of the embodiments herein, under the same conditions, the molecules comprising one or more mismatches can be less stably bound to the target nucleic acid than the molecules comprising no mismatch in the interrogatory region. In any one of the embodiments herein, the method can comprise one or more stringency washes. For instance, one or more stringency washes can be used to remove probe molecules that are not bound to the target nucleic acid, and/or probe molecules that are bound to the target nucleic acid but comprise one or more mismatches in the interrogatory region.

Following formation of, e.g., the circularized probe, in some instances, an amplification primer is added. In other instances, the amplification primer is added with the circularizable probes. In some instances, the amplification primer may also be complementary to the target nucleic acid and the circularizable probe. In some embodiments, a washing step is performed to remove any unbound probes, primers, etc. In some embodiments, the wash is a stringency wash. Washing steps can be performed at any point during the process to remove non-specifically bound probes, probes that have ligated, etc. In some embodiments, the stringency is increased in the hybridization of the probe or probe set to the target nucleic acid, reducing or negating the need of performing a stringency wash.

Upon addition of a DNA polymerase in the presence of appropriate dNTP precursors and other cofactors, the amplification primer is elongated by replication of multiple copies of the template (e.g., a concatemer of the template is generated). This amplification product can be detected using, e.g., the secondary and higher order probes and detection oligonucleotides described herein. In some embodiments, the sequence of the amplicon or a portion thereof, is determined or otherwise analyzed, for example by using detectably labeled probes and imaging. The sequencing or analysis of the amplification products can comprise sequencing by hybridization, sequencing by ligation, and/or fluorescent in situ sequencing, and/or wherein the in situ hybridization comprises sequential fluorescent in situ hybridization. In some instances, sequencing using, e.g., the secondary and higher order probes and detection oligonucleotides described herein.

In any one of the embodiments herein, the method can further comprise generating the product of the circularized probe in situ in the biological sample. In any one of the embodiments herein, the product can be generated using rolling circle amplification (RCA). In any one of the embodiments herein, the RCA can comprise a linear RCA, a branched RCA, a dendritic RCA, or any combination thereof. In any one of the embodiments herein, the product can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (such as, amplicon) containing multiple copies of the cDNA. Techniques for rolling circle amplification (RCA) include but are not limited to linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, an exemplary workflow for analyzing a biological sample, comprises contacting the biological sample with a circularizable probe, wherein: the probe comprises a first hybridization region capable of hybridizing to a first target sequence in a target nucleic acid in the biological sample, a second hybridization region capable of hybridizing to a second target sequence in the target nucleic acid and a stem-loop structure at the 3′ or 5′ end of the circularizable probe, wherein upon hybridization of the circularizable probe to the target nucleic acid, the stem-loop structure is positioned between the first and second hybridization regions; cleaving the circularizable probe with a nuclease to generate a ligatable 3′ end or ligatable 5′ end and release the stem-loop structure or a portion thereof; ligating the ligatable 3′ end or ligatable 5′ end to the 5′ end or the 3′ end of the circularizable probe, respectively, to generate a circular probe using the target nucleic acid as template; amplifying the circular probe using rolling circle amplification (RCA) to generate an RCA product; and detecting the RCA product in the biological sample.

In some embodiments, an exemplary workflow for analyzing a biological sample, comprises contacting the target RNA with a first probe and a second probe simultaneously or in any order, wherein the first probe comprises a first hybridization region capable of hybridizing to a first target RNA sequence in the target RNA, and a stem-loop structure, the second probe comprises a second hybridization region capable of hybridizing to a second target RNA sequence in the target RNA, and upon hybridization of the first and second probes to the target RNA, the stem-loop structure is at positioned between the first and second hybridization regions; cleaving the first probe with a nuclease to generate a first ligatable end, thereby releasing the stem-loop structure or a portion thereof; ligating the first ligatable end to a second ligatable end in the second probe to generate a ligated probe using the target RNA as template; and detecting the ligated probe or a product thereof. In some embodiments, target RNA is in a cell or tissue sample and the ligated probe and/or the product thereof is generated in the cell or tissue sample. In some embodiments, the ligated probe and/or the product thereof is detected in the cell or tissue sample.

In some embodiments, an exemplary workflow for analyzing a biological sample, comprises contacting the biological sample with a circularizable probe comprising a first hybridization region capable of hybridizing to a first target sequence in a target nucleic acid in the biological sample, a second hybridization region capable of hybridizing to a second target sequence in the target nucleic acid, a stem-loop structure at the 3′ or 5′ end of the circularizable probe, wherein the target nucleic acid comprises a region of interest and the stem-loop structure comprises an interrogatory region, and wherein upon hybridization of the circularizable probe to the target nucleic acid, the stem-loop structure is positioned between the first and second hybridization regions; cleaving the circularizable probe with a nuclease to generate a ligatable 3′ end or ligatable 5′ end and releasing the stem-loop structure or a portion thereof, thereby allowing the interrogatory region to hybridize to the region of interest. In some aspects, if the interrogatory region is complementary to the region of interest, ligating the ligatable 3′ end or ligatable 5′ end to the 5′ end or the 3′ end of the circularizable probe, respectively, to generate a circular probe using the target nucleic acid as template; and amplifying the circular probe using rolling circle amplification (RCA) to generate an RCA product; and detecting the RCA product in the biological sample. In some embodiments, the target nucleic acid is an RNA and the ligating step is performed using a ligase having an RNA-templated ligase activity. In some embodiments, the interrogatory region is in the stem-loop structure or a region on the 3′ or the 5′ of the stem-loop structure, and the interrogatory region is not in the first or second hybridization region. In some embodiments, the interrogatory region is in the stem of the stem-loop structure, the loop of the stem-loop structure, or a single-stranded region between the stem-loop structure and the first or second hybridization region. In some embodiments, the region of interest is between the first and second target sequences. In some embodiments, the interrogatory region is in the first hybridization region and the region of interest is in the first target sequence, and the stem-loop structure blocks the circularization of the circularizable probe until the stem-loop structure or portion thereof is released. In some embodiments, the interrogatory region is in the second hybridization region and the region of interest is in the second target sequence, and the stem-loop structure blocks the circularization of the circularizable probe until the stem-loop structure or portion thereof is released. In some embodiments, further comprising prior to the ligating step, a step of removing molecules of the circularizable probe that are not bound to the target nucleic acid from the biological sample. In some embodiments, further comprising prior to the ligating step, a step of removing molecules of the circularizable probe that are bound to the target nucleic acid but comprise one or more mismatches in the interrogatory region from the biological sample, and/or allowing the molecules comprising one or more mismatches to dissociate from the target nucleic acid while the molecules comprising no mismatch in the interrogatory region remain bound to the target nucleic acid. In some embodiments, under the same conditions, the molecules comprising one or more mismatches are less stably bound to the target nucleic acid than the molecules comprising no mismatch in the interrogatory region.

### VI. Spatial Array Capture

In some embodiments, after the nuclease-driven cleavage of the duplex region and the generation of ligatable ends of the probe or probe sets described in Sections IV-V, the assay may further comprise one or more optional steps for transferring the probes (or a product or derivative thereof) to an array. In some embodiments, the probes (e.g., first probe and second probe) can be ligated and then transferred to an array. In some embodiments, a product (e.g., extension product) or derivative of the ligated probes (e.g., ligated first-second probe) can be transferred to an array.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array. Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

In some cases, spatial analysis can be performed by attaching and/or introducing a molecule having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. Some such methods of spatial analysis are described in Section (III) of WO 2020/176788 and/or U.S. Patent Application Publication No. 2020/0277663.

In some cases, spatial analysis can be performed by detecting multiple oligonucleotides (e.g., probe or probe set as described in Section III) that hybridize to a target nucleic acid (e.g., comprising a region of interest). In some instances, for example, spatial analysis can be performed by hybridization of two oligonucleotides (e.g., a first probe and a second probe as described in Section III) to adjacent sequences on an analyte (e.g., an RNA molecule, such as an mRNA molecule). In some instances, the oligonucleotides (e.g., a circularizable probe or probe set as described in Section III) are DNA molecules. In some instances, one of the oligonucleotides (e.g., a circularizable probe or probe set) includes at least two ribonucleic acid bases at the 3′ end and/or the other oligonucleotide includes a phosphorylated nucleotide at the 5′ end. In some instances, one of two of the oligonucleotides (e.g., (e.g., a circularizable probe or probe set)) includes a duplex region or a stem-loop structure. In some instances, one of the two oligonucleotides (e.g., (e.g., a circularizable probe or probe set)) includes a capture domain (e.g., a poly(A) sequence, a non-homopolymeric sequence). In some instances, one or both of the oligonucleotides (e.g., a circularizable probe or probe set) may comprise an interrogatory region. Cleavage of the duplex region or the stem-loop structure generates 3′ and/or 5′ ligatable ends. Sequence complementarity between an interrogatory region in one or both of the oligonucleotides (e.g., a circularizable probe or probe set) and a sequence of interest (e.g., an SNP) in the target nucleic acid facilitates stable hybridization of the two oligonucleotides (e.g., a circularizable probe or probe set) to the target nucleic acid in or associated with the analyte. After hybridization to the analyte, and cleavage of the duplex region or the stem-loop structure, a ligase (e.g., SplintR ligase) ligates the two oligonucleotides (e.g., a circularizable probe or probe set) together, creating a ligation product (e.g., ligated circularized probe or ligated first-second probe). In some instances, the two oligonucleotides (e.g., a circularizable probe or probe set) hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product (or a derivative thereof) can then be captured by an oligonucleotide (e.g., a capture probe immobilized, directly or indirectly, on a substrate) comprising a capture sequence complementary to a sequence of the ligated probe(s) on an array, optionally amplified, and sequenced, thus determining the location and optionally the abundance of the analyte in the biological sample. In some embodiments, the ligated probe(s) comprise a complementary capture sequence. In some instances, a ligated probe comprises an overhang region (e.g., a region that does not hybridize to the target nucleic acid) comprising the complementary capture sequence. In some embodiments, the capture sequence comprises a polyA sequence. In some embodiments, the oligonucleotide (e.g., capture probe) comprising the capture sequence comprises a spatial barcode sequence. In some embodiments, the method comprises generating a spatially barcoded oligonucleotide comprising (i) a sequence of the ligated probe or product therof or complement thereof and (ii) a sequence of the spatial barcode sequence or complement thereof.

In some embodiments, after ligating the first ligatable end to a second ligatable end in the circularizable probe or probe set, the method can comprise a step of removing unligated molecules of the probe or probe set (e.g., first and/or second probes that are not ligated). In some embodiments, ligation of the circularizable probe or probe set (e.g., the first and second probe) stabilizes hybridization of the probe(s) to the target nucleic acid. In some embodiments, the method can comprise one or more stringency washes to remove unligated probes (optionally, prior to migrating probes to capture oligonucleotides).

In some instances, the cleaved duplex region or stem-loop structure or a fragment thereof, can be migrated toward an oligonucleotide comprising a capture sequence (e.g., a capture probe immobilized, directly or indirectly, on a substrate) on an array, optionally amplified, and sequenced to determine the spatial location of a molecule of interest (for example, an mRNA molecule). In some instances, the cleavage product migration toward the capture probe can take place prior to and/or during probe ligation (e.g., ligation of a circularizable probe or probe set after cleaving off a duplex region). After hybridization of the duplex region, the stem-loop structure, or a fragment thereof to a capture probe, the duplex region, stem-loop structure, or fragment thereof is extended to make a copy of the additional components (for example, the spatial barcode) of the capture probe.

A capture probe can be any molecule capable of capturing (directly or indirectly) a circularizable probe, probe set or product thereof corresponding to an analyte in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, at least one of the probes for binding a target nucleic acid (e.g., probe or probe set as described in Section III) comprise a sequence complementary to a sequence comprised by an oligonucleotide comprising a capture sequence (e.g. capture probe). In some embodiments, a plurality of the probes (e.g., first or second probes, or ligated probes generated from the first and second probes) comprise a common sequence for hybridizing to a capture sequence on the oligonucleotide (e.g. capture probe). In some embodiments, the capture probes are spatially barcoded capture probes attached to a substrate (e.g., array). In some instances, the spatially barcoded capture probes described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer binding sequence (such as an R1, R2, or partial R1 or R2 sequence). In some embodiments, the method comprises generating a spatially barcoded oligonucleotide comprising (i) a sequence of the ligated probe or product therof or complement thereof and (ii) a sequence of the spatial barcode sequence or complement thereof. In some examples, a ligation product (or a derivative thereof) can be captured by the oligonucleotides comprising the capture sequence (e.g. capture probes) and associated with a spatial barcode, optionally amplified, and sequenced, thus determining the location of the target nucleic acid. In some cases, the spatially barcoded oligonucleotides can be attached to functional sequences described herein (such as sequence specific flow cell attachment sequences) prior to analysis. In some cases, the spatially barcoded oligonucleotide (or a product or derivative thereof) can be released from the substrate (e.g., array) prior to analysis. In some cases, the spatially barcoded oligonucleotide can be further processed and subjected to one or more reactions prior to analysis (e.g., extension, amplification, or other reactions described in Section V). In some embodiments, the spatially barcoded oligonucleotides are detected by nucleic acid sequencing. In some other embodiments, the spatially barcoded oligonucleotides are detected at the spatial location in the biological sample.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially barcoded array (e.g., including spatially barcoded capture probes). Another method is to cleave spatially barcoded capture probes from an array and promote the spatially barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

In some embodiments, an extended capture probe can be a capture probe having additional nucleotides added to the terminus (e.g., 3′ or 5′ end) of the capture probe thereby extending the overall length of the capture probe. For example, an extended 3′ end can comprise additional nucleotides added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Typically, for spatial array-based methods, a substrate functions as a support for direct or indirect attachment of capture probes to features of the array. A feature can be an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or corresponding probes (e.g., described in Section III and processed as described in Sections IV-V) can be captured when contacting a biological sample with a substrate including capture probes (e.g., a substrate with capture probes embedded, spotted, printed, fabricated on the substrate, or a substrate with features (e.g., wells) comprising capture probes). In the context of spatial array capture, contacting a biological sample with a substrate can comprise any contact (e.g., direct or indirect) such that capture probes can interact (e.g., bind covalently or non-covalently (e.g., hybridize)) with analytes from the biological sample. In some aspects, the oligonucleotide comprises a capture sequence complementary to a sequence of the ligated probe and/or the product thereof. In some embodiments, a target nucleic acid is at a location in a biological sample, the ligated probe is generated at the location in the biological sample, and the ligated probe and/or product thereof is covalently or noncovalently attached to an oligonucleotide (e.g., capture probe) immobilized on a substrate. In other embodiments, a target nucleic acid is covalently or noncovalently attached to an oligonucleotide (e.g., capture probe) immobilized on a substrate and the ligated probe and/or product thereof is generated. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

During analysis of spatial information, sequence information for a spatial barcode associated with an analyte (e.g., ligated first-second probe or product or derivative thereof) is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes (or corresponding probes or products generated therefrom) from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655, US 20220062246, and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

In some embodiments, an exemplary workflow for analyzing a biological sample, comprises contacting the target RNA with a first probe and a second probe simultaneously or in any order, wherein the first probe comprises a first hybridization region capable of hybridizing to a first target RNA sequence in the target RNA, and a stem-loop structure, the second probe comprises a second hybridization region capable of hybridizing to a second target RNA sequence in the target RNA, and upon hybridization of the first and second probes to the target RNA, the stem-loop structure is at positioned between the first and second hybridization regions; cleaving the first probe with a nuclease to generate a first ligatable end, thereby releasing the stem-loop structure or a portion thereof; ligating the first ligatable end to a second ligatable end in the second probe to generate a ligated probe using the target RNA as template; and detecting the ligated probe or a product thereof. In some embodiments, target RNA is in a cell or tissue sample and the ligated probe and/or the product thereof is generated in the cell or tissue sample. In some embodiments, the ligated probe and/or the product thereof is covalently or noncovalently attached to an oligonucleotide immobilized on a substrate. In some embodiments, the oligonucleotide comprises a spatial barcode sequence and a spatially barcoded oligonucleotide comprising (i) a sequence of the ligated probe or product thereof or complement thereof and (ii) a sequence of the spatial barcode sequence or complement thereof is generated and sequenced.

### VII. Detection and Analysis

In some aspects, after formation of a hybridization complex comprising nucleic acid probes and/or probe sets described in Section III and any one or more further processing steps (e.g., ligation, extension amplification, capture, or any combination thereof) as described in Sections IV-VI, the method can further include detection of the probe or probe set hybridized to the target nucleic acid or any products generated therefrom or a derivative thereof (for example, the cleaved duplex region). In any one of the embodiments herein, the method can further comprise imaging the biological sample to detect a ligation product or a circularized probe or product thereof. In any one of the embodiments herein, a sequence of the ligation product, rolling circle amplification product, or other generated product can be analyzed in situ in the biological sample. In any one of the embodiments herein, the imaging can comprise detecting a signal associated with a fluorescently labeled probe that directly or indirectly binds to a rolling circle amplification product of the circularized probe. In any one of the embodiments herein, the sequence of the ligation product, rolling circle amplification product, extension product, or other generated product can be analyzed by sequential hybridization, sequencing by hybridization, sequencing by ligation, sequencing by synthesis, sequencing by binding, or a combination thereof. In some cases, a spatially barcoded analyte (or a product or derivative thereof) can be released from an array prior to analysis.

In any one of the embodiments herein, a sequence associated with the target nucleic acid or the probe(s) can comprise one or more barcode sequences or complements thereof. In any one of the embodiments herein, the sequence of the rolling circle amplification product can comprise one or more barcode sequences or complements thereof. In any one of the embodiments herein, a ligated linear probe (e.g., generated from a first and second probe described herein) can comprise one or more barcode sequences or complements thereof. In some embodiments, a ligated linear probe can comprise an overhang region (e.g., a region that does not hybridize to the target nucleic acid) comprising one or more barcode sequences or complements thereof, which can be detected according to any of the methods described herein (optionally, wherein the detection comprises signal amplification). In some embodiments, a ligated linear probe can be released from the target nucleic acid (e.g., by RNase H digestion) prior to detecting a sequence of the ligated linear probe. In any one of the embodiments herein, the one or more barcode sequences can comprise a barcode sequence corresponding to the target nucleic acid. In any one of the embodiments herein, the one or more barcode sequences can comprise a barcode sequence corresponding to the sequence of interest, such as variant(s) of a single nucleotide of interest.

In some aspects, any of the probe(s) described herein can comprise one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or UMI). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some embodiments, barcodes or complements thereof (e.g., barcode sequences or complements thereof comprised by the probes disclosed herein or products thereof) can be analyzed (e.g., detected or sequenced) using any suitable method or technique, including those described herein, such as sequencing by synthesis (SBS), sequencing by ligation (SBL), or sequencing by hybridization (SBH). In some instances, barcoding schemes and/or barcode detection schemes as described in RNA sequential probing of targets (RNA SPOTs), single-molecule fluorescent in situ hybridization (smFISH), multiplexed error-robust fluorescence in situ hybridization (MERFISH) or sequential fluorescence in situ hybridization (seqFISH+) can be used. In any of the preceding implementations, the methods provided herein can include analyzing the barcodes by sequential hybridization and detection with a plurality of labelled probes (e.g., detection probes (e.g., detection oligos) or barcode probes). In some instances, the barcode detection steps can be performed as described in hybridization-based in situ sequencing (HybISS). In some instances, probes can be detected and analyzed (e.g., detected or sequenced) as performed in fluorescent in situ sequencing (FISSEQ), or as performed in the detection steps of the spatially-resolved transcript amplicon readout mapping (STARmap) method. In some instances, signals associated with an analyte can be detected as performed in sequential fluorescent in situ hybridization (seqFISH).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and 20210164039, which are hereby incorporated by reference in their entirety.

In any one of the embodiments herein, instead of detecting the presence of oligonucleotides (e.g., circularizable probes or probe sets) that hybridize directly to a target nucleic acid, the methods disclosed herein include detecting the cleavage of a non-hybridized sequence of an oligonucleotide (for example, a duplex region) and further analyzing to determine the presence of absence of an analyte (for example, an mRNA molecule) of interest. In any one of the embodiments herein, the method includes determining all or part of the duplex region. In any one of the embodiments herein, the duplex region further includes a barcode sequence. When the duplex is cleaved, the duplex can be used to identify a hybridization and/or ligation event where the circularizable probe or the first and second nucleic acid probe undergo ligation. In some aspects, the determining all or part of the duplex region includes sequencing. In some instances, the duplex region can include a functional sequence. The functional sequence can be a primer sequence. The primer sequence can be used to amplify the duplex region or the stem-loop structure before cleavage, contemporaneously with cleavage, or after cleavage of the duplex region from the nucleic acid probe.

In any one of the embodiments herein, the target nucleic acid is at a location in a biological sample and the ligated probe is generated at the location in the biological sample, and the ligated probe and/or the product thereof is detected at the location in the biological sample.

In any one of the embodiments herein, the detecting step can comprise contacting the biological sample with one or more detectably-labeled probes that directly or indirectly hybridize to the rolling circle amplification product, and dehybridizing the one or more detectably-labeled probes from the rolling circle amplification product. In any one of the embodiments herein, the contacting and dehybridizing steps can be repeated with the one or more detectably-labeled probes and/or one or more other detectably-labeled probes that directly or indirectly hybridize to the rolling circle amplification product. In preferred embodiments, the detectably-labeled probes directly hybridize to the rolling circle amplification product (e.g., generated as described in Section V).

In any one of the embodiments herein, the detecting step can comprise contacting the biological sample with one or more intermediate probes that directly or indirectly hybridize to the rolling circle amplification product, wherein the one or more intermediate probes are detectable using one or more detectably-labeled probes. In any one of the embodiments herein, the detecting step can further comprise dehybridizing the one or more intermediate probes and/or the one or more detectably-labeled probes from the rolling circle amplification product. In any one of the embodiments herein, the contacting and dehybridizing steps can be repeated with the one or more intermediate probes, the one or more detectably-labeled probes, one or more other intermediate probes, and/or one or more other detectably-labeled probes.

In some embodiments, the detection may be spatial, e.g., in two or three dimensions. In some embodiments, the detection may be quantitative, e.g., the amount or concentration of a primary nucleic acid probe (and of a target nucleic acid) or a stem-loop structure may be determined. In some embodiments, the primary probes, secondary probes, higher order probes, and/or detectably labeled probes may comprise any one of a variety of entities able to hybridize a nucleic acid, e.g., DNA, RNA, LNA, and/or PNA, etc., depending on the application.

In some embodiments, disclosed herein is a multiplexed assay where multiple targets (e.g., nucleic acids such as genes or RNA transcripts, or protein targets) are probed with multiple primary probes (e.g., circularizable primary probes), and optionally multiple secondary probes hybridizing to the primary barcodes (or complementary sequences thereof) are all hybridized at once, followed by sequential secondary barcode detection and decoding of the signals. In some embodiments, detection of barcodes or subsequences of the barcode can occur in a cyclic manner.

In some embodiments, a method for analyzing a region of interest in a target nucleic acid is a multiplexed assay where multiple probes (e.g., circularizable probes) are used to detect multiple regions of interest simultaneously (e.g., variations at the same location of a target nucleic acid and/or SNPs in various locations). In some embodiments, one or more detections of one or more regions of interest may occur simultaneously. In some embodiments, one or more detections of one or more regions of interest may occur sequentially. In some embodiments, multiple circularizable probes of the same circularizable probe design are used to detect one or more regions of interest, using different barcodes associated with each region of interest. In some embodiments, multiple circularizable probes of different circularizable probe design are used to detect one or more regions of interest, using different barcodes (e.g., each barcode associated with a target nucleic acid or sequence thereof). In some embodiments, the one or more regions of interest are localized on the same molecule (e.g., RNA or DNA). In alternative embodiments, the one or more single nucleotides of interest are localized on different molecules.

In some aspects, the provided methods involve analyzing, e.g., detecting or determining, one or more sequences present in the polynucleotides (e.g., probe or probe set) and/or in a product or derivative thereof, such as in an amplified circularized probe. In some embodiments, the detection comprises providing detection probes, such as probes for performing a chain reaction that forms an amplification product, e.g., HCR. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region of interest in a target nucleic acid. In other embodiments, the provided methods involve analyzing, e.g., detecting or determining, one or more sequences present in the polynucleotide probes (e.g., a barcode sequence or present in a probe or product thereof) or a cleaved stem-loop structure or a fragment thereof.

In some embodiments, a method disclosed herein may also comprise one or more signal amplification components. In some embodiments, the present disclosure relates to the detection of nucleic acids sequences in situ using probe hybridization and generation of amplified signals associated with the probes, wherein background signal is reduced and sensitivity is increased. In some embodiments, the RCA product generated using a method disclosed herein can be detected in with a method that comprises signal amplification.

Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594, the content of which is herein incorporated by reference in its entirety), hybridization chain reaction, assembly of topologically catenated DNA structures using serial rounds of chemical ligation (clampFISH), signal amplification via hairpin-mediated concatemerization (e.g., as described in US 2020/0362398 the content of which is herein incorporated by reference in its entirety), e.g., primer exchange reactions such as signal amplification by exchange reaction (SABER) or SABER with DNA-Exchange (Exchange-SABER). In some embodiments, a non-enzymatic signal amplification method may be used.

The detectable reactive molecules may comprise tyramide, such as used in tyramide signal amplification (TSA) or multiplexed catalyzed reporter deposition (CARD)-FISH. In some embodiments, the detectable reactive molecule may be releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, US 2019/0376956, WO 2020/102094, US 2022/0026433, WO 2020/163397, US 2022/0128565, and WO 2021/067475, all of which are incorporated herein by reference in their entireties.

In some embodiments, hybridization chain reaction (HCR) can be used for signal amplification. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an initiator nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g., initiator or other HCR monomer) when the monomers are in the hairpin structure may be referred to as the toehold region (or input domain). The first HCR monomers each further comprise a sequence which is complementary to a sequence in the exposed toehold region of the second HCR monomers. This sequence of complementarity in the first HCR monomers may be referred to as the interacting region (or output domain). Similarly, the second HCR monomers each comprise an interacting region (output domain), e.g., a sequence which is complementary to the exposed toehold region (input domain) of the first HCR monomers. In the absence of the HCR initiator, these interacting regions are protected by the secondary structure (e.g., they are not exposed), and thus the hairpin monomers are stable or kinetically trapped (also referred to as metastable), and remain as monomers (e.g., preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g., the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived. Branching HCR systems have also been devised and described (see, e.g., WO 2020/123742 and US 20220064697, all of which are herein incorporated by reference in their entireties), and may be used in the methods herein.

In some embodiments, similar to HCR reactions that use hairpin monomers, linear oligo hybridization chain reaction (LO-HCR) can also be used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. In any one of the embodiments herein, the target nucleic acid molecule and/or the analyte can be an RCA product.

In some embodiments, detection of nucleic acids sequences in situ includes combination of RCA with an assembly for branched signal amplification. In some embodiments, the assembly complex comprises an amplifier hybridized directly or indirectly (via one or more oligonucleotides) to a sequence of the RCA product. In some embodiments, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. No. US20200399689A1 and Xia et al., Multiplexed Detection of RNA using MERFISH and branched DNA amplification. Scientific Reports (2019), each of which is entirely incorporated by reference herein.

In some embodiments, the RCA product can be detected with a method that comprises signal amplification by performing a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with a sample comprising RCA products generated using methods described herein. In various embodiments, the RCA product may be contacted with a plurality of concatemer primers and a plurality of labeled probes. see e.g., U.S. Pat. Pub. No. US20190106733, the content of which is herein incorporated by reference in its entirety, for exemplary molecules and PER reaction components.

In some embodiments, the methods comprise sequencing all or a portion of the amplification product, such as one or more barcode sequences present in the amplification product. In some embodiments, extended capture probes can be amplified (e.g., in bulk solution or on the array) and analyzed, e.g., via DNA sequencing.

In some embodiments, the product or derivative of a first and second probe ligated together after hybridizing to the target nucleic acid can be analyzed by sequencing. In some embodiments, the analysis and/or sequence determination comprises sequencing all or a portion of the amplification product or the probe(s) and/or in situ hybridization to the amplification product or the probe(s). In some embodiments, the sequencing step involves sequencing by hybridization, sequencing by ligation, and/or fluorescent in situ sequencing, hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction, see e.g., US 2017/0009278, which is the content of which is herein incorporated by reference in its entirety, for exemplary probes and HCR reaction components. In some embodiments, the detection or determination comprises hybridizing to the amplification product a detection oligonucleotide labeled with a fluorophore, an isotope, a mass tag, or a combination thereof. In some embodiments, the detection or determination comprises imaging the amplification product. In some embodiments, the target nucleic acid is an mRNA in a tissue sample, and the detection or determination is performed when the target nucleic acid and/or the amplification product is in situ in the tissue sample.

In some aspects, provided herein are in situ assays using microscopy as a readout, e.g., nucleic acid sequencing, hybridization, or other detection or determination methods involving an optical readout. In some aspects, detection or determination of a sequence of one, two, three, four, five, or more nucleotides of a target nucleic acid is performed in situ in a cell in an intact tissue. In some aspects, the detection or determination is of a sequence associated with or indicative of a target nucleic acid. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product). In some embodiments, a method for spatially profiling analytes such as the transcriptome or a subset thereof in a biological sample is provided. Methods, compositions, kits, devices, and systems for these in situ assays, comprising spatial genomics and transcriptomics assays, are provided. In some embodiments, a provided method is quantitative and preserves the spatial information within a tissue sample without physically isolating cells or using homogenates. In some embodiments, the present disclosure provides methods for high-throughput profiling one or more single nucleotides of interest in a large number of targets in situ, such as transcripts and/or DNA loci, for detecting and/or quantifying nucleic acids in cells, tissues, organs or organisms.

In some aspects, the provided methods comprise imaging the amplification product (e.g., amplicon) and/or one or more portions of the polynucleotides, for example, via binding of the detection probe and detecting the detectable label. In some embodiments, the detection probe comprises a detectable label that can be measured and quantitated. A label or detectable label can be a directly or indirectly detectable moiety that is associated with (e.g., conjugated to) a molecule to be detected, e.g., a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. Autofluorescence can comprise background fluorescence (that can arise from a variety of sources, including aldehyde fixation, extracellular matrix components, red blood cells, lipofuscin, and the like), which is distinct from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label can be used to detect one or more polynucleotide(s) and/or amplification products (e.g., amplicon) described herein. In some embodiments, the methods involve incubating the detectable probe containing the detectable label with the sample, washing unbound detectable probe, and detecting the label, e.g., by imaging.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as 1251, 35S, 14C, or 3H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. A fluorescent label can comprise a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-!2-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Nucleotides having other fluorophores can also be synthesized (See, Henegariu et al. (2000) Nature Biotechnol. 18:345).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. In some embodiments, the term antibody comprises an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6xHis), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or an polynucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and 5,192,782. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any one of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequences can be analyzed in situ, e.g., by incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (i.e., nucleotide sequence) of the incorporated nucleotides or labeled primer extension products can be determined, and consequently, the nucleotide sequence of the corresponding template nucleic acid. Aspects of in situ analysis are described, for example, in Mitra et al., (2003) Anal. Biochem. 320, 55-65, and Lee et al., (2014) Science, 343(6177), 1360-1363; US 2016/0024555; US 2019/0194709; U.S. Pat. Nos. 10,138,509; 10,494,662; 10,179,932.

In some cases, sequencing can be performed after the analytes are released from the biological sample. In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/005986, US 2005/0100900, U.S. Pat. No. 9,217,178, US 2009/0118128, US 2012/0270305, US 2013/0260372, and US 2013/0079232.

In some embodiments, sequencing can be performed by sequential fluorescence hybridization (e.g., sequencing by hybridization). Sequential fluorescence hybridization can involve sequential hybridization of detection probes comprising an oligonucleotide and a detectable label.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597.

In some embodiments, the barcodes of the probes (e.g., the circularizable probe or a spatially barcoded analyte comprising a sequence of the ligated probes) or complements or products thereof are targeted by detectably labeled detection oligonucleotides, such as fluorescently labeled oligonucleotides. In some embodiments, one or more decoding schemes are used to decode the signals, such as fluorescence, for sequence determination. In any one of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., Science; 348(6233):aaa6090 (2015); Gyllborg et al., Nucleic Acids Res (2020) 48(19):e112; U.S. Pat. No. 10,457,980 B2; US 2016/0369329 A1; US 2021/0017587; and US 2017/0220733 A1, all of which are incorporated by reference in their entireties. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004).

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

### VIII. Single Cell Profiling

Disclosed herein, are methods and systems for characterizing nucleic acids from small populations of cells, and in some cases, for characterizing nucleic acids from individual cells, especially in the context of larger populations of cells. Particulars of the steps of the methods can be carried out as described herein, for example in Sections III-V and VII; and/or using any suitable processes and methods for carrying out the particular steps.

In one aspect, the methods and systems described herein, provide for the compartmentalization, depositing or partitioning of the nucleic acid contents of individual cells from a sample material containing cells, into discrete compartments or partitions (referred to interchangeably herein as partitions), where each partition maintains separation of its own contents from the contents of other partitions. Unique identifiers, e.g., barcodes, may be previously, subsequently or concurrently delivered to the partitions that hold the compartmentalized or partitioned cells, in order to allow for the later attribution of the characteristics of the individual cells to the particular compartment. These partitions may be comprised of, e.g., microcapsules or micro-vesicles that have an outer barrier surrounding an inner fluid center or core, or they may be a porous matrix that is capable of entraining and/or retaining materials within its matrix. In some aspects, however, these partitions comprise droplets of aqueous fluid within a non-aqueous continuous phase, e.g., an oil phase. A variety of different vessels are described in, for example, U.S. Patent Publication No. 2014/0155295, the full disclosure of which is incorporated herein by reference in its entirety for all purposes. Likewise, emulsion systems for creating stable droplets in non-aqueous or oil continuous phases are described in detail in, e.g., U.S. Patent Publication No. 2010/0105112, the full disclosure of which is incorporated herein by reference in its entirety for all purposes.

In many cases, the systems and methods are used to ensure that the substantial majority of occupied partitions (partitions containing one or more microcapsules) include no more than 1 cell per occupied partition. In some cases, the partitioning process is controlled such that fewer than 25% of the occupied partitions contain more than one cell, and in many cases, fewer than 20% of the occupied partitions have more than one cell, while in some cases, fewer than 10% or even fewer than 5% of the occupied partitions include more than one cell per partition.

As will be appreciated, the above-described occupancy rates are also applicable to partitions that include both cells and supports (e.g., beads) carrying the barcode oligonucleotides. In particular, in some aspects, a substantial percentage of the overall occupied partitions will include both a bead and a cell.

In some aspects, the cells may be partitioned along with lysis reagents in order to release the contents of the cells within the partition. In such cases, the lysis agents can be contacted with the cell suspension concurrently with, or immediately prior to the introduction of the cells into the partitioning junction/droplet generation zone, e.g., through an additional channel or channels upstream of channel junction. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Similarly, lysis methods that employ other methods may be used, such as electroporation, thermal, acoustic or mechanical cellular disruption may also be used in certain cases, e.g., non-emulsion based partitioning such as encapsulation of cells that may be in addition to or in place of droplet partitioning, where any pore size of the encapsulate is sufficiently small to retain nucleic acid fragments of a desired size, following cellular disruption.

In addition to the lysis agents co-partitioned with the cells described above, other reagents can also be co-partitioned with the cells, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated cells, the cells may be exposed to an appropriate stimulus to release the cells or their contents from a co-partitioned microcapsule. For example, in some cases, a chemical stimulus may be co-partitioned along with an encapsulated cell to allow for the degradation of the microcapsule and release of the cell or its contents into the larger partition. In some cases, this stimulus may be the same as the stimulus described elsewhere herein for release of oligonucleotides from their respective bead or partition. In alternative aspects, this may be a different and non-overlapping stimulus, in order to allow an encapsulated cell to be released into a partition at a different time from the release of oligonucleotides into the same partition.

In some embodiments, the partition is a microwell or a droplet (e.g., emulsion droplet). In some embodiments, a discrete droplet that is generated may include an individual biological particle (e.g., a cell). A discrete droplet that is generated may include a barcode carrying support (e.g., bead). A discrete droplet generated may include both an individual biological particle and a barcode carrying bead, such as droplets. In some instances, a discrete droplet may include more than one individual biological particle or no biological particle. In some instances, a discrete droplet may include more than one bead or no bead. A bead may be porous, non-porous, solid, semi-solid, semi-fluidic, fluidic, and/or any combination thereof. In some instances, a bead may be dissolvable, disruptable, and/or degradable. In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible. A bead may be of any suitable shape. Examples of bead shapes include, but are not limited to, spherical, non-spherical, oval, oblong, amorphous, circular, cylindrical, and variations thereof.

In some examples, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide), which may include a priming sequence (e.g., a primer for amplifying target nucleic acids, random primer, primer sequence for messenger RNA) and/or one or more barcode sequences. The one more barcode sequences may include sequences that are the same for all nucleic acid molecules (e.g., oligonucleotides) coupled to a given bead and/or sequences that are different across all oligonucleotides coupled to the given bead. The oligonucleotides may be incorporated into the bead. For example, a partition comprises a support (e.g., bead) that comprises a plurality fo barcoded oligonucleotides comprising a partition barcode sequence and a capture sequence. In some cases, the oligonucleotides can comprise a functional sequence, for example, for attachment to a sequencing flow cell, such as, for example, a P5 sequence for Illumina® sequencing. In some cases, the oligonucleotides or derivative thereof (e.g., barcoded oligonucleotide or polynucleotide generated from the barcoded oligonucleotides) can comprise another functional sequence, such as, for example, a P7 sequence for attachment to a sequencing flow cell for Illumina sequencing. In some cases, the oligonucleotides can comprise a barcode sequence. In some cases, the primer can further comprise a unique molecular identifier (UMI). In some cases, the primer can comprise a R1 primer sequence for Illumina sequencing. In some cases, the primer can comprise an R2 primer sequence for Illumina sequencing. Examples of such oligonucleotides (e.g., barcoded oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

Additional reagents may also be co-partitioned with the cells, such as endonucleases to fragment the cell's DNA, DNA polymerase enzymes and dNTPs used to amplify the cell's nucleic acid fragments and to attach the barcode oligonucleotides to the amplified fragments. Additional reagents may also include reverse transcriptase enzymes, including enzymes with terminal transferase activity, primers and oligonucleotides, and switch oligonucleotides (or “switch oligos”) which can be used for template switching. In some cases, template switching can be used to increase the length of a cDNA. In one example of template switching, cDNA can be generated from reverse transcription of a template, e.g., cellular mRNA, where a reverse transcriptase with terminal transferase activity can add additional nucleotides, e.g., polyC, to the cDNA that are not encoded by the template, such, as at an end of the cDNA. Switch oligos can include sequences complementary to the additional nucleotides, e.g., polyG. The additional nucleotides (e.g., polyC) on the cDNA can hybridize to the sequences complementary to the additional nucleotides (e.g., polyG) on the switch oligo, whereby the switch oligo can be used by the reverse transcriptase as template to further extend the cDNA. Switch oligos may comprise deoxyribonucleic acids, ribonucleic acids, modified nucleic acids including locked nucleic acids (LNA), or any combination.

In some embodiments, additional reagents may include probes or probe sets comprising duplex regions (for example as described in Section III) and nucleases. In some embodiments, the nucleases cleave sites within the duplex regions thereby releasing the duplex region and generating ligatable 3′ and 5′ ends. In some embodiments, additional reagents such as those disclosed in Sections III-V, include DNA polymerases, dNTPs, ligases, that facilitate extension, ligation and amplification of the cleaved circularizable probe or probe sets.

Once the contents of the cells are released into their respective partitions, the nucleic acids contained therein may be further processed within the partitions. In accordance with the methods and systems described herein, the nucleic acid contents of individual cells are generally provided with unique identifiers such that, upon characterization of those nucleic acids they may be attributed as having been derived from the same cell or cells. The ability to attribute characteristics to individual cells or groups of cells is provided by the assignment of unique identifiers specifically to an individual cell or groups of cells, which is another advantageous aspect of the methods and systems described herein. The oligonucleotides are partitioned such that as between oligonucleotides in a given partition, the nucleic acid barcode sequences contained therein are the same, but as between different partitions, the oligonucleotides can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

The co-partitioned oligonucleotides can also comprise other functional sequences useful in the processing of the nucleic acids from the co-partitioned cells. These sequences include, e.g., targeted or random/universal amplification primer sequences for amplifying the genomic DNA from the individual cells within the partitions while attaching the associated barcode sequences, sequencing primers or primer recognition sequences, hybridization or probing sequences, e.g., for identification of presence of the sequences or for pulling down barcoded nucleic acids, or any of a number of other potential functional sequences. Again, co-partitioning of oligonucleotides and associated barcodes and other functional sequences, along with sample materials is described in, for example, U.S. Patent Application Nos. 61/940,318, filed Feb. 7, 2014, 61/991,018, filed May 9, 2014, and U.S. patent application Ser. No. 14/316,383, filed Jun. 26, 2014, as well as U.S. patent application Ser. No. 14/175,935, filed Feb. 7, 2014, the full disclosures of which are incorporated herein by reference in their entireties for all purposes.

As will be appreciated, a number of other reagents may be co-partitioned along with the cells, beads, lysis agents and chemical stimuli, including, for example, protective reagents, like proteinase K, chelators, nucleases, ligation, nucleic acid extension, replication, transcription or amplification reagents such as polymerases, reverse transcriptases, nucleoside triphosphates or NTP analogues, primer sequences and additional cofactors such as divalent metal ions used in such reactions, ligation reaction reagents, such as ligase enzymes and ligation sequences, dyes, labels, or other tagging reagents.

Briefly, in one aspect, the probes or probe sets comprising duplex regions as disclosed herein (e.g., in Section III) are contacted with a sample of cells that has been fixed and permeabilized. The probes and/or probe sets (e.g., a linear probe set, as shown in FIG. 1B) then hybridize to the plurality of target mRNA sequences (for example, mRNA sequences of the single cell that is partitioned). In some embodiments, the probe or probe set comprises: i) a first hybridization region capable of hybridizing to a first target sequence in a target nucleic acid in the biological sample, and ii) a second hybridization region capable of hybridizing to a second target sequence in the target nucleic acid, and iii) a duplex region wherein upon hybridization of the probe or probe set to the target nucleic acid, the duplex region is positioned between the first and second hybridization regions. In any of the embodiments herein, the method can further comprise a wash step to remove excess and unbound probes. In some cases, partitioning is performed such that nucleic acid contents and any probe(s) bound thereto of individual cells from the sample material containing cells are provided into discrete compartments or partitions where each partition maintains separation of its own contents from the contents of other partitions along with beads carrying the barcode oligonucleotides. In some aspects, the cells may be partitioned along with lysis reagents in order to release the contents of the cells within the partition and other reagents such as nucleases (e.g., release the duplex region), ligases to generate the ligated probes (e.g., ligated first-second probe), DNA polymerase enzymes and dNTPs used to amplify the cell's nucleic acid fragments and to attach the barcode oligonucleotides to the amplified fragments. In some embodiments, a nuclease disclosed herein cleaves the probe or probe set to generate a ligatable end(s) thereby releasing the duplex region. In some embodiments, the 3′ and 5′ ends of a first and second probe can be ligated to generate a ligated first-second probe in the partition (e.g., droplets). In any of the embodiments herein, the target nucleic acid is from a cell that is partitioned and the ligated probe is generated in the partition.

In the partitions, barcoding of the ligated probe(s) and/or products thereof may take place using the barcode oligonucleotides. For example, each of the beads comprises an oligonucleotide (e.g., a barcoded capture oligonucleotide) comprising a barcode (e.g., partition barcode sequence), a UMI, and a capture sequence for hybridizing to a complementary capture sequence on the ligated probes (e.g., ligated first-second probe). In some instances, a ligated probe comprises an overhang region (e.g., a region that does not hybridize to the target nucleic acid) comprising the complementary capture sequence. In some embodiments, the capture sequence comprises a polyA sequence. After cells are partitioned into aqueous droplets in an emulsion, along with beads linked to oligonucleotides comprising a barcode sequence, a UMI sequence, and a capture sequence, barcoded ligated probe molecules comprising a sequence of the ligated probes are generated by extension of the captured ligated probe using the barcoded capture oligonucleotide as a template. Thus, the resulting products (e.g., barcoded ligated probe molecules) comprise a sequence of the ligated probe or product thereof, or a complement of the ligated probe or product thereof, tagged with the barcode (e.g., a sequence of the partition barcode sequence of complement therof) and UMI from the barcoded capture oligonucleotide (e.g., barcoded oligonucleotide). In some embodiments, the resulting barcodeed ligated probe molecules that are barcoded can be released from the support, collected, amplified, and/or further processed for downstream sequencing. The barcoded ligated probe molecules and/or amplified products can be detected by nucleic acid sequencing. In some embodiments, the barcoded oligonucleotides (e.g., barcoded ligated probe molecules) or products thereof for sequencing comprise a sequence of the ligated probe or product thereof or complement thereof and a sequence of the partition barcode sequence or complement thereof.

In some embodiments, a plurality of barcoded oligonucleotides (e.g., barcoded-ligated probe molecules) are released and collected from a plurality of partitions and pooled (e.g., pooled with contents of other partitions) prior to further processing (shearing, attachment of functional sequences, and subsequent amplification (e.g, via PCR)), and these operations may occur in bulk (e.g, outside the partition). In the case where a partition is a droplet in an emulsion, the emulsion can be broken and the contents of the droplet pooled in order to perform subsequent processing (e.g., amplification, attachment of other functional sequences). In some cases, barcoded oligonucleotide can be initially associated with the support (e.g., by releasably attaching to the support) and then released. Release of the barcoded oligonucleotides can be passive. In addition or alternatively, release from the support can be upon application of a stimulus which allows the barcoded oligonucleotides to dissociate or to be released from the support. Such stimulus may disrupt the bead, and such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof. Once pooled, a solution may be a pooled mixture comprising the contents of two or more partitions (e.g., droplets).

All of the fragments from multiple different partitions may then be pooled for sequencing on high throughput sequencers as described herein, where the pooled fragments comprise a large number of fragments derived from the nucleic acids of different cells or small cell populations, but where the fragments from the nucleic acids of a given cell will share the same barcode sequence. In particular, because each fragment is coded as to its partition of origin, and consequently its single cell or small population of cells, the sequence of that fragment may be attributed back to that cell or those cells based upon the presence of the barcode, which will also aid in applying the various sequence fragments from multiple partitions to assembly of individual genomes for different cells.

In some embodiments, an exemplary workflow for analyzing a target nucleic acid comprises (a) providing a partition comprising (i) a single biological particle, wherein said single biological particle comprises the target nucleic acid and a probe or probe set hybridized to the target nucleic acid, and (ii) a bead comprising a plurality of barcode oligonucleotides each comprising a partition barcode sequence and capture sequence complementary to a sequence in the probe or probe set or a product thereof; wherein the probe or probe set comprises: i) a first hybridization region capable of hybridizing to a first target sequence in the target nucleic acid, ii) a second hybridization region capable of hybridizing to a second target sequence in the target nucleic acid, and iii) a duplex region, wherein upon hybridization of the probe or probe set to the target nucleic acid, the duplex region is positioned between the first and second hybridization regions; wherein the probe or probe set is cleaved with a nuclease to generate a first ligatable end and release the duplex region or a portion thereof; b) ligating the first ligatable end to a second ligatable end in the probe or probe set hybridized to the target nucleic acid to generate a ligated probe; and (c) using a barcode oligonucleotide of the plurality of the barcode oligonucleotides to generate a barcoded ligated probe molecule comprising (i) a sequence of the ligated probe or product thereof or complement thereof and (ii) a sequence of the partition barcode sequence or complement thereof; (d) releasing the barcoded ligated probe molecule from the partition and amplifying the barcoded ligated probe molecule; and (e) determining a sequence of the barcoded ligated probe molecule or a portion thereof. In some embodiments, the biological particle is a cell. In some embodiments, determining the sequence of the barcoded ligated probe molecule or a portion thereof comprises performing next generation sequencing.

In some embodiments, an exemplary workflow for analyzing a biological sample, comprises contacting the target RNA with a first probe and a second probe simultaneously or in any order, wherein the first probe comprises a first hybridization region capable of hybridizing to a first target RNA sequence in the target RNA, and a stem-loop structure, the second probe comprises a second hybridization region capable of hybridizing to a second target RNA sequence in the target RNA, and upon hybridization of the first and second probes to the target RNA, the stem-loop structure is at positioned between the first and second hybridization regions; cleaving the first probe with a nuclease to generate a first ligatable end, thereby releasing the stem-loop structure or a portion thereof; ligating the first ligatable end to a second ligatable end in the second probe to generate a ligated probe using the target RNA as template; and detecting the ligated probe or a product thereof. In some embodiments, the target RNA is in a partition, and the partition comprises a single cell or components of the single cell containing the target RNA. In some embodiments, a ligated probe or product thereof is generated in the partition, and the partition further comprises a support that comprises a nucleic acid molecule comprising a partition barcode sequence. In some embodiments, a barcoded oligonucleotide comprising (i) a sequence of the ligated probe or product thereof or complement thereof and (ii) a sequence of the partition barcode sequence or complement thereof is generated and sequenced. In some examples, a generated barcoded oligonucleotide comprising (i) a sequence of the ligated probe or product thereof or complement thereof and (ii) a sequence of the partition barcode sequence or complement thereof can be released from the partition, collected with other generated barcoded oligonucloeitdes and processed in bulk for downstream sequencing.

### IX. Compositions, Kits, and Systems

In some embodiments, disclosed herein is a composition that comprises a complex containing a target nucleic acid, a probe, e.g., any one of the probes described herein, and a probe or probe set comprising a duplex region (for example, within a stem-loop structure). In some embodiments, the complex further comprises a nuclease cleavage site for the generation of ligatable probes.

In some embodiments, disclosed herein is a composition that comprises an amplification product containing monomeric units of a sequence complementary to a sequence of a probe (e.g., a circularizable probe). In some embodiments, the amplification product is formed using any one of the target nucleic acids, probes (e.g., circularizable probes) and any one of the amplification techniques described herein.

Also provided herein are kits, for example comprising one or more polynucleotides, e.g., any described in Section III, and reagents for performing the methods provided herein, for example reagents required for one or more steps comprising hybridization, cleavage, ligation, amplification, detection, sequencing, and/or sample preparation as described herein. In some embodiments, the kit further comprises a target nucleic acid. In some embodiments, any or all of the polynucleotides are DNA molecules. In some embodiments, the target nucleic acid is a messenger RNA molecule. In some embodiments, the kit further comprises a nuclease, for instance for cleaving the duplex region (for example, duplex region within a stem-loop structure). In some embodiments, the nuclease is a restriction endonuclease. In some embodiments, the nuclease is a nickase. In some embodiments, the kit further comprises a ligase, for instance for forming a ligated circularized probe from the circularizable probe (e.g., padlock probe) or probe set. In some embodiments, the ligase has DNA-splinted DNA ligase activity. In some embodiments, the kit further comprises a polymerase, for instance for performing amplification of the circularizable probe. In some embodiments, the polymerase is capable of using the ligated circular probe as a template for amplification. In some embodiments, the kit further comprises a primer for amplification.

In some embodiments, the kits may further comprise one or more reagents for array based analysis, such as capture probes, e.g., any described in Section VI. For example, provided are kits comprising a substrate (e.g., an array) with a plurality of oligonucleotides immobilized thereon. For example, the oligonucleotides immobilized on the substrate each comprise a spatial barcode sequence and a capture sequence complementary to a sequence of the ligated probe and/or the product thereof (e.g., described inm Section III-V).

In some embodiments, the kits may further comprise one or more reagents for single cell analysis, such as for partitioning and barcoding nucleic acids, e.g., any described in Section VIII. For example, a kit comprises a plurality of beads each comprising a plurality of barcode oligonucleotides. In some instances, the barcode oligonucleotides each comprise a partition barcode sequence and capture sequence complementary to a sequence in a probe or probe set or a product thereof (e.g., as described in Sections III-V). In some embodiments, the kits may further include reagents for amplification and sequencing of a barcoded ligated probe molecule.

Disclosed herein in some aspects is a kit comprising a probe or probe set, and the probe or probe set comprises: i) a first hybridization region capable of hybridizing to a first target sequence in a target nucleic acid, ii) a second hybridization region capable of hybridizing to a second target sequence in the target nucleic acid, and iii) a duplex region, wherein upon hybridization of the probe or probe set to the target nucleic acid, the duplex region is positioned between the first and second hybridization regions.

Disclosed herein in some aspects is a kit comprising a circularizable probe for use in a method for detecting a region of interest in a target nucleic acid, the method comprising: a) contacting the biological sample with the circularizable probe comprising: i) a first hybridization region capable of hybridizing to a first target sequence in a target nucleic acid in the biological sample, ii) a second hybridization region capable of hybridizing to a second target sequence in the target nucleic acid, iii) a stem-loop structure at the 3′ or 5′ end of the circularizable probe, wherein the target nucleic acid comprises a region of interest and the stem-loop structure comprises an interrogatory region, and wherein upon hybridization of the circularizable probe to the target nucleic acid, the stem-loop structure is positioned between the first and second hybridization regions; b) cleaving the circularizable probe with a nuclease to generate a ligatable 3′ end or ligatable 5′ end and releasing the stem-loop structure or a portion thereof, thereby allowing the interrogatory region to hybridize to the region of interest; c) if the interrogatory region is complementary to the region of interest, ligating the ligatable 3′ end or ligatable 5′ end to the 5′ end or the 3′ end of the circularizable probe, respectively, to generate a circular probe using the target nucleic acid as template; and d) amplifying the circular probe using rolling circle amplification (RCA) to generate an RCA product; and e) detecting the RCA product in the biological sample.

In some aspects, disclosed herein is a kit comprising a plurality of circularizable probes for use in a method for analyzing a biological sample comprising a plurality of target RNA molecules comprising a single nucleotide of interest, the method comprising: a) contacting the biological sample with a plurality of circularizable probes, each comprising: i) a first hybridization region capable of hybridizing to a first target sequence in the target mRNA molecule, ii) a second hybridization region capable of hybridizing to a second target sequence in the target mRNA molecule, iii) a stem-loop structure at the 3′ or 5′ end of the circularizable probe, wherein the target nucleic acid comprises a region of interest and the stem-loop structure comprises an interrogatory region, and wherein upon hybridization of the circularizable probe to the target nucleic acid, the stem-loop structure is positioned between the first and second hybridization regions; b) cleaving the circularizable probe with a nuclease to generate a ligatable 3′ end or ligatable 5′ end and releasing the stem-loop structure or a portion thereof, thereby allowing the interrogatory region to hybridize to the region of interest; c) if the interrogatory region is complementary to the region of interest, ligating the ligatable 3′ end or ligatable 5′ end to the 5′ end or the 3′ end of the circularizable probe, respectively, to generate a circular probe using the target nucleic acid as template; and d) amplifying the circular probe using rolling circle amplification (RCA) to generate an RCA product; and e) detecting the RCA product in the biological sample.

In some aspects, disclosed herein is a kit for use in a method for analyzing a biological sample, the method comprising: a) contacting the biological sample with a probe or probe set, wherein the probe or probe set comprises: i) a first hybridization region capable of hybridizing to a first target sequence in a target nucleic acid in the biological sample, ii) a second hybridization region capable of hybridizing to a second target sequence in the target nucleic acid, and iii) a duplex region, wherein upon hybridization of the probe or probe set to the target nucleic acid, the duplex region is positioned between the first and second hybridization regions; b) cleaving the probe or probe set with a nuclease to generate a first ligatable end and releasing the duplex region or a portion thereof; c) ligating the first ligatable end to a second ligatable end in the probe or probe set to generate a ligated probe; and d) detecting the ligated probe or a product thereof in the biological sample.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any one of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

### X. Applications

In some aspects, the provided embodiments can be applied in an in situ method of analyzing nucleic acid sequences, such as an in situ transcriptomic analysis, for example from intact tissues or samples in which the spatial information has been preserved. In some aspects, the embodiments can be applied in a spatial array. In some aspects, the embodiments can be applied in a single-cell profiling assay. In some aspects, the embodiments can be applied in an imaging or detection method for multiplexed nucleic acid analysis. In some aspects, the provided embodiments can be used to identify or detect regions of interest in target nucleic acids.

In some embodiments, the region of interest comprises more than one nucleotide of interest. In some embodiments, the region of interest is a single nucleotide of interest. In some embodiments, the single nucleotide of interest is a single-nucleotide polymorphism (SNP). In some embodiments, the single nucleotide of interest is a single-nucleotide variant (SNV). In some embodiments, the single nucleotide of interest is a single-nucleotide substitution. In some embodiments, the single nucleotide of interest is a point mutation. In some embodiments, the single nucleotide of interest is a single-nucleotide insertion. In some embodiments, the single nucleotide of interest is a single-nucleotide deletion.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### XI. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.”

A “hybridization complex” as used herein may comprise one, two, or more strands or separate molecules. A hybridization complex that comprises three or more strands or separate molecules does not necessarily comprise direct hybridization between every possible pairwise combination thereof, so long as at least two molecules or strands are directly hybridized to each other, or are in the process of binding to or unbinding from each other, at a given time.

“Hybridization conditions” typically include salt concentrations of approximately less than 1 M, often less than about 500 mM and may be less than about 200 mM. A “hybridization buffer” includes a buffered salt solution such as 5% SSPE, or other such buffers. Hybridization temperatures can be as low as 5° C., but are typically greater than 22° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. Several equations for calculating the Tₘ of nucleic acids can be used. As indicated by standard references, a simple estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any one of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1x SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, for instance, where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif.; HeliScope™ by Helicos Biosciences Corporation, Cambridge, Ma.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

“SNP” or “single nucleotide polymorphism” may include a genetic variation between individuals; e.g., a single nitrogenous base position in the DNA of organisms that is variable. SNPs are found across the genome; much of the genetic variation between individuals is due to variation at SNP loci, and often this genetic variation results in phenotypic variation between individuals. SNPs for use in the present disclosure and their respective alleles may be derived from any number of sources, such as public databases (U.C. Santa Cruz Human Genome Browser Gateway (genome.ucsc.edu/cgi-bin/hgGateway) or the NCBI dbSNP website (www.ncbi.nlm.nih gov/SNP/), or may be experimentally determined as described in U.S. Pat. No. 6,969,589; and US Pub. No. 2006/0188875 entitled “Human Genomic Polymorphisms.” Although the use of SNPs is described in some of the embodiments presented herein, it will be understood that other biallelic or multi-allelic genetic markers may also be used. A biallelic genetic marker is one that has two polymorphic forms, or alleles. As mentioned above, for a biallelic genetic marker that is associated with a trait, the allele that is more abundant in the genetic composition of a case group as compared to a control group is termed the “associated allele,” and the other allele may be referred to as the “unassociated allele.” Thus, for each biallelic polymorphism that is associated with a given trait (e.g., a disease or drug response), there is a corresponding associated allele. Other biallelic polymorphisms that may be used with the methods presented herein include, but are not limited to multinucleotide changes, insertions, deletions, and translocations. It will be further appreciated that references to DNA herein may include genomic DNA, mitochondrial DNA, episomal DNA, and/or derivatives of DNA such as amplicons, RNA transcripts, cDNA, DNA analogs, etc. The polymorphic loci that are screened in an association study may be in a diploid or a haploid state and, ideally, would be from sites across the genome.

“Multiplexing” or “multiplex assay” herein may refer to an assay or other analytical method in which the presence and/or amount of multiple targets, e.g., multiple nucleic acid target sequences, can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “adjacent” as used herein includes but is not limited to being directly linked by a phosphodiester bond. For example, “adjacent” nucleotides or regions on a nucleic acid such as a probe may be separated by a number of nucleotides. For instance, a toehold region and an interrogatory region adjacent to each other may be separated by 0, 1, 2, 3, 4, 5, 6, 7, 8, 9, 10 or more nucleotides in a probe.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

The term “each,” when used in reference to a collection of items, is intended to identify an individual item in the collection but does not necessarily refer to every item in the collection, unless expressly stated otherwise, or unless the context of the usage clearly indicates otherwise.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Design of Probes

This Example describes the design of a circularizable (e.g., padlock) probe for in situ, single cell, and spatial analysis applications. The probe or probe set disclosed herein permits improved ligation stringency of RNA templated ligation reactions (such as ligation of DNA probes based on an RNA-template) for accurate RNA downstream sequencing or detection. The probe or probe set comprises a 3′ or 5′ end sequence that does not hybridize to the target nucleic acid, such as messenger RNA (mRNA) in the biological sample but requires an additional exonuclease or a restriction enzyme step to cleave the 3′ or 5′ end sequence and allow for ligation of the circularizable DNA padlock probe. The design of the probe or probe set thus ensures that only those probes that are hybridized to the target RNA and are processed by the exonuclease or restriction enzyme will be ligatable.

FIGS. 1 and 2 depict schematics of an exemplary probe and probe set. The probe in FIG. 1 comprises a circularizable probe with at least two hybridization regions that are capable of hybridizing to at least two adjacent complementary regions on a target nucleic acid (for example, to the target nucleic acid, such as an mRNA). The circularizable probe also contains a duplex region which includes a first and second strand hybridized to one another. The duplex region can also be modified to comprise a stem-loop structure. The duplex region or stem-loop structure can be positioned at either the 3′ end (FIG. 1) or the 5′ end of the circularizable probe, or can be positioned internally (for example, positioned between the hybridization region and the 3′ or 5′ end). In some cases, the duplex region runs continuous between the first and second hybridization regions such that the probe is circular (FIG. 2B).

In some instances, a probe set comprising a set of two linear probes are hybridized to two complementary regions on a target nucleic acid (for example, to the target nucleic acid, such as an mRNA, or to another hybridization region in a polynucleotide of the probe set). In this instance, the duplex can be positioned at either the 3′ or 5′ end of the first or second linear probe of the probe set (FIG. 2C). The duplex can also be positioned between the two probes such that the two probes are connected to each other to form a single linearized probe.

The duplex region or stem-loop structure can contain a restriction endonuclease recognition sequence. The restriction endonuclease can cleave the site generating either blunt or sticky ends. In some instances, the restriction endonuclease can cleave at a site outside the recognition sequence (for example, in the case of type IIS enzymes). In some cases, the nuclease that cleaves the duplex region or stem-loop structure can be a uracil-specific excision reagent enzyme. In some cases, the nuclease can be a nickase, which cleaves only one strand of the duplex region.

### Example 2: Hybridization, Cleavage, Ligation, Amplification and Detection

Exemplary probe and probe sets as described in Example 1 above are hybridized, cleaved, ligated, amplified and detected in an exemplary sample for transcriptional profiling of isolated cells or in an intact biological sample, such as a tissue section.

A library of different probe or probe sets targeting different target nucleic acids, or different probe sets targeting the same nucleic acid at different positions, are applied with hybridization buffers to the biological sample. The sample is washed and incubated with a nuclease (such as a restriction endonuclease, uracil-specific excision reagent enzyme, or a nickase) for cleavage of the duplex region or stem-loop structure. The released duplex or stem-loop structure is then washed using a wash buffer. The buffer conditions used allow the probe or probe set to remain hybridized to the target nucleic acid. The sample is then incubated with a ligase (such as a T4 DNA ligase) for the ligation of the ligatable 3′ and 5′ ends of the probe or probe set. Only those probe or probe sets that have been processed by the nuclease to generate ligatable ends, can undergo ligation. The probe or probe sets that are not processed remain partially hybridized and are removed in a subsequent stringency wash. The sample is then incubated with a rolling-circle amplification (RCA) mixture containing a DNA polymerase and dNTPs, and incubated for amplification of the ligated probes.

For in situ detection, the amplified product generated using the ligated probes or probe sets is detected using detectably (e.g., fluorescently) labelled oligonucleotides complementary to a portion of the amplified probe product. For example, the amplification product has multiple copies of barcode sequences for detection. The samples are washed and images are obtained. Multiple cycles of hybridization and imaging can be performed.

For sequencing, the sample is treated with a stripping buffer, washed, and treated with a sequencing mixture containing a T4 DNA ligase, fluorescently labeled sequencing oligonucleotide, and images are obtained. Multiple cycles of sequencing are performed, and DAPI and Nissl staining can also be performed. Images are acquired using a confocal microscope.

The assay can also be multiplexed to analyze a plurality of amplification products from different mRNA molecules to spatially profile the transcriptome or a subset thereof in the biological sample. Once the probes comprising the duplex region are cleaved and ligated in situ, tissues are optionally permeated with Proteinase K (ProK) and/or a de-crosslinking step can be optionally performed such that molecules such as mRNAs, cDNAs, probes, ligation products, and/or amplification products are no longer locked in place (e.g., to a hydrogel). In some cases, the probes or products thereof are migrated onto an array comprising spatially barcoded capture probes. In some instances, the ligated probe or product thereof or complement thereof or amplified probe (such as a circularized probe) comprises a sequence that can be captured by the capture probes of the array-slide. The tissue is lysed and the probe or product thereof or complement thereof is captured onto the array-slide for attachment of a spatial barcode sequence to generate a plurality of spatially barcoded oligonucleotides. Library preparation and sequencing is then performed.

### Example 3: Use of a Padlock Probe with a Stem-Loop Structure to Increase Ligation Specificity and Stringency when Detecting a Region of Interest in a Target mRNA Molecule

This Example describes the design and use of a circularizable probe, such as a padlock probe, comprising a duplex region or stem-loop structure for detecting a region of interest in a target nucleic acid, such as an SNP in a messenger RNA.

In the example shown in FIG. 3, the probe is a modified padlock probe for detecting an SNP, comprising a duplex region (such as a stem-loop structure) at the 3′ or 5′ end of the padlock probe. Such an approach can be used to increase specificity and stringency when detecting a region of interest (such as an SNP) in a target nucleic acid, such as an mRNA. The padlock probe comprises at least two hybridization regions that hybridize to complementary sequences on the target mRNA. The padlock probe comprises an interrogatory region complementary to the region of interest, at the 3′ or 5′ end of the probe. The interrogatory region connects the 3′ or 5′ end of the padlock probe to the stem-loop structure in such a manner that upon cleavage and release of the stem-loop structure, and generation of a ligatable end, the interrogatory region is free to hybridize to the region of interest in the target mRNA.

A mixture of probes is incubated with hybridization buffer for hybridization of the probes to target nucleic acid (such as mRNA) in the sample. The sample is washed and incubated with a nuclease (such as a restriction endonuclease, uracil-specific excision reagent enzyme, or a nickase) for cleavage of the stem-loop structure. The released stem-loop structure is then removed using a wash buffer. The buffer conditions used allow the probe to remain hybridized to the target nucleic acid. As shown in FIG. 3, if the complementary SNP is not present, the padlock probe would still bind the mRNA target to the complementary regions. However, the mismatched end, for example the 3′ end, will not hybridize to the region of interest in the mRNA and the ligation will be halted. Because the arm of the padlock probe is not fully hybridized, the padlock would be removed in a stringency wash. If the interrogatory region is complementary to the region of interest in the target mRNA, such that the padlock probe will be stably hybridized to the target, ligation will proceed. The sample is then incubated at room temperature with a T4 DNA ligase for ligation of the ligatable 3′ and 5′ ends of the padlock probes to form circularized probes. A primer for amplification of the circularized probe may be added. The sample is then incubated with a rolling-circle amplification (RCA) mixture containing a Phi29 DNA polymerase and dNTP for RCA of the circular probes. Fluorescently labeled oligonucleotides complementary to a portion of the RCA product, a barcode contained therein, or a secondary probe attached thereto are incubated with the sample. Multiple cycles of contacting the sample with probes and sequence determination (e.g., using in situ sequencing based on sequencing-by-ligation or sequencing-by-hybridization) can be performed. Fluorescent images can be obtained in each cycle, and one or more wash steps can be performed in a cycle or between cycles. Probe targeting various SNPs within or across genes can be sequentially or simultaneously provided, processed, and detected as described above.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for analyzing a target nucleic acid, comprising:
a) contacting the target nucleic acid with a probe or probe set comprising:
i) a first hybridization region capable of hybridizing to a first target sequence in the target nucleic acid,
ii) a second hybridization region capable of hybridizing to a second target sequence in the target nucleic acid, and
iii) a duplex region,
wherein upon hybridization of the probe or probe set to the target nucleic acid, the duplex region is positioned between the first and second hybridization regions;
b) cleaving the probe or probe set with a nuclease to generate a first ligatable end and release the duplex region or a portion thereof;
c) ligating the first ligatable end to a second ligatable end in the probe or probe set hybridized to the target nucleic acid to generate a ligated probe; and
d) detecting the ligated probe or a product thereof.

2-4. (canceled)

5. The method of claim 1, wherein the duplex region is at a 3′ end or at a 5′ end of the probe or a probe in the probe set.

6-7. (canceled)

8. The method of claim 1, wherein the probe or probe set comprises a stem-loop structure comprising the duplex region.

9-10. (canceled)

11. The method of claim 1, wherein the duplex region comprises a first strand and a second strand and the first strand and the second strand are in the same probe molecule comprising self-complementary sequences.

12. The method of claim 1, wherein the duplex region comprises a first strand and a second strand and the first strand is at the 5′ end and the second strand is at the 3′ end of the probe molecule or vice versa.

13-14. (canceled)

15. The method of claim 1, wherein the duplex region comprises a first strand and a second strand and the first strand is at the 5′ end of a first probe and the second strand is at the 3′ end of a second probe, or wherein the first strand is at the 3′ end of the first probe and the second strand is at the 5′ end of the second probe.

16-18. (canceled)

19. The method of claim 1, wherein the cleaving comprises cleaving a site in the duplex region.

20-24. (canceled)

25. The method of claim 1, wherein the probe or probe set comprises a non-ligatable end and the cleaving in b) removes the non-ligatable end.

26. The method of claim 25, wherein the non-ligatable end comprises a 3′ dideoxynucleotide, lacks a 3′ hydroxyl group, or is 5′ dephosphorylated.

27. The method of claim 1, wherein the ligated probe is a linear probe.

28. The method of claim 1, wherein the ligated probe is a circular probe.

29. The method of claim 1, wherein the target nucleic acid is at a location in a biological sample and the ligated probe is generated at the location in the biological sample, and wherein the ligated probe and/or the product thereof is detected at the location in the biological sample.

30-55. (canceled)

56. The method of claim 1, wherein the nuclease is a restriction endonuclease.

57. The method of claim 56, wherein cleavage by the restriction endonuclease generates blunt ends.

58. (canceled)

59. The method of claim 56, wherein cleavage by the restriction endonuclease generates sticky ends.

60-62. (canceled)

63. The method of claim 1, wherein the nuclease is a uracil-specific excision reagent enzyme.

64. The method of claim 1, wherein the nuclease is a nickase.

65. The method of claim 64, wherein the nickase is selected from the group consisting of Nt.CviPII, Nb.BsmI, Nb.BbvCI, Nb.BsrDI, Nb.BtsI, Nt.BsmAI, Nt.BbvCI, NtBspQI, Nt.AlwI, and Nt.BstNBI.

66-89. (canceled)

90. A method for analyzing a biological sample, comprising:
a) contacting the biological sample with a probe comprising:
i) a first hybridization region capable of hybridizing to a first target sequence in a target nucleic acid in the biological sample,
ii) a second hybridization region capable of hybridizing to a second target sequence in the target nucleic acid, and
iii) a stem-loop structure at the 3′ or 5′ end of the probe,
wherein upon hybridization of the probe to the target nucleic acid, the stem-loop structure is positioned between the first and second hybridization regions;
b) cleaving the probe with a nuclease to generate a ligatable 3′ end or ligatable 5′ end and release the stem-loop structure or a portion thereof;
c) ligating the ligatable 3′ end or ligatable 5′ end to the 5′ end or the 3′ end of the probe, respectively, to generate a circular probe using the target nucleic acid as template;
d) amplifying the circular probe using rolling circle amplification (RCA) to generate an RCA product in the biological sample; and
e) detecting the RCA product.

91. (canceled)

92. A method for analyzing a biological sample, comprising:
a) contacting the biological sample with a probe comprising:
i) a first hybridization region capable of hybridizing to a first target sequence in a target nucleic acid in the biological sample,
ii) a second hybridization region capable of hybridizing to a second target sequence in the target nucleic acid,
iii) a stem-loop structure at the 3′ or 5′ end of the probe,
wherein the target nucleic acid comprises a region of interest and the probe comprises an interrogatory region, and
wherein upon hybridization of the probe to the target nucleic acid, the stem-loop structure is positioned between the first and second hybridization regions;
b) cleaving the probe with a nuclease to generate a ligatable 3′ end or ligatable 5′ end and release the stem-loop structure or a portion thereof;
c) if the interrogatory region is complementary to the region of interest, ligating the ligatable 3′ end or ligatable 5′ end to the 5′ end or the 3′ end of the probe, respectively, to generate a circular probe using the target nucleic acid as template; and
d) amplifying the circular probe using rolling circle amplification (RCA) to generate an RCA product in the biological sample; and
e) detecting the RCA product.

93-101. (canceled)
